SEQUENCE LISTING (147 sequences)

SEQ ID NO: 1 - protein, 330 aa, Homo sapiens [sequence omitted]

SEQ ID NO: 2 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - protein, 107 aa, Homo sapiens [sequence omitted]

SEQ ID NO: 4 - protein, 104 aa, Homo sapiens
PKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 5 - DNA, 21 bp, Homo sapiens
  source 1..21: mol_type = genomic DNA
tcaccatggactgcacctgga

SEQ ID NO: 6 - DNA, 21 bp, Homo sapiens
  source 1..21: mol_type = genomic DNA
tcaccatggactggacctgga

SEQ ID NO: 7 - DNA, 22 bp, Homo sapiens
  source 1..22: mol_type = genomic DNA
ccatggacacactttgctccac

SEQ ID NO: 8 - DNA, 22 bp, Homo sapiens
  source 1..22: mol_type = genomic DNA
ccatggacacactttgttccac

SEQ ID NO: 9 - DNA, 23 bp, Homo sapiens
  source 1..23: mol_type = genomic DNA
tcaccatggagtttgggctgagc

SEQ ID NO: 10 - DNA, 25 bp, Homo sapiens
  source 1..25: mol_type = genomic DNA
agaacatgaaacacctgtggttctt

SEQ ID NO: 11 - DNA, 25 bp, Homo sapiens
  source 1..25: mol_type = genomic DNA
agaacatgaaacatctgtggttctt

SEQ ID NO: 12 - DNA, 20 bp, Homo sapiens
  source 1..20: mol_type = genomic DNA
atggggtcaaccgccatcct

SEQ ID NO: 13 - DNA, 23 bp, Homo sapiens
  source 1..23: mol_type = genomic DNA
acaatgtctgtctccttcctcat

SEQ ID NO: 14 - DNA, 19 bp, synthetic construct
  misc_feature 1..19: note = huCH-II
  source 1..19: mol_type = other DNA
gccagggggaagaccgatg

SEQ ID NO: 15 - DNA, 19 bp, Homo sapiens
  source 1..19: mol_type = genomic DNA
gccagggggaagacggatg

SEQ ID NO: 16 - DNA, 21 bp, Homo sapiens
  source 1..21: mol_type = genomic DNA
gctcagctcctggggctcctg

SEQ ID NO: 17 - DNA, 21 bp, Homo sapiens
  source 1..21: mol_type = genomic DNA
ctggggctgctaatgctctgg

SEQ ID NO: 18 - DNA, 21 bp, Homo sapiens
  source 1..21: mol_type = genomic DNA
ttcctcctgctactctggctc

SEQ ID NO: 19 - DNA, 21 bp, Homo sapiens
  source 1..21: mol_type = genomic DNA
cagacccaggtcttcatttct

SEQ ID NO: 20 - DNA, 24 bp, Homo sapiens
  source 1..24: mol_type = genomic DNA
tttcaactgctcatcagatggcgg

SEQ ID NO: 21 - DNA, 20 bp, Homo sapiens
  source 1..20: mol_type = genomic DNA
cctctcctcctcaccctcct

SEQ ID NO: 22 - DNA, 18 bp, Homo sapiens
  source 1..18: mol_type = genomic DNA
ctcctcactcagggcaca

SEQ ID NO: 23 - DNA, 19 bp, Homo sapiens
  source 1..19: mol_type = genomic DNA
atggcctggatccctctcc

SEQ ID NO: 24 - DNA, 19 bp, Homo sapiens
  source 1..19: mol_type = genomic DNA
atggcctggacccctctcc

SEQ ID NO: 25 - DNA, 19 bp, Homo sapiens
  source 1..19: mol_type = genomic DNA
atggcctggatcgctctcc

SEQ ID NO: 26 - DNA, 19 bp, Homo sapiens
  source 1..19: mol_type = genomic DNA
atggcctggaccgctctcc

SEQ ID NO: 27 - DNA, 20 bp, Homo sapiens
  source 1..20: mol_type = genomic DNA
agctcctcagaggagggcgg

SEQ ID NO: 28 - DNA, 20 bp, Homo sapiens
  source 1..20: mol_type = genomic DNA
agctcctcagaggagggtgg

SEQ ID NO: 29 - DNA, 38 bp, Homo sapiens
  source 1..38: mol_type = genomic DNA
ctttaagaaggagatataccatggtgcagctggtgcag

SEQ ID NO: 30 - DNA, 38 bp, Homo sapiens
  source 1..38: mol_type = genomic DNA
ctttaagaaggagatataccatggttcagctggtgcag

SEQ ID NO: 31 - DNA, 41 bp, Homo sapiens
  source 1..41: mol_type = genomic DNA
ctttaagaaggagatataccatgcaggtccagcttgtgcag

SEQ ID NO: 32 - DNA, 41 bp, Homo sapiens
  source 1..41: mol_type = genomic DNA
ctttaagaaggagatataccatggaggtccagctggtacag

SEQ ID NO: 33 - DNA, 41 bp, Homo sapiens
  source 1..41: mol_type = genomic DNA
ctttaagaaggagatataccatgcaggtccagctggtacag

SEQ ID NO: 34 - DNA, 41 bp, Homo sapiens
  source 1..41: mol_type = genomic DNA
ctttaagaaggagatataccatgcaaatgcagctggtgcag

SEQ ID NO: 35 - DNA, 41 bp, Homo sapiens
  source 1..41: mol_type = genomic DNA
ctttaagaaggagatataccatgcagatgcagctggtgcag

SEQ ID NO: 36 - DNA, 41 bp, Homo sapiens
  source 1..41: mol_type = genomic DNA
ctttaagaaggagatataccatgcagatcaccttgaaggag

SEQ ID NO: 37 - DNA, 41 bp, Homo sapiens
  source 1..41: mol_type = genomic DNA
ctttaagaaggagatataccatgcaggtcaccttgaaggag

SEQ ID NO: 38 - DNA, 41 bp, Homo sapiens
  source 1..41: mol_type = genomic DNA
ctttaagaaggagatataccatgcaggtcaccttgagggag

SEQ ID NO: 39 - DNA, 41 bp, Homo sapiens
  source 1..41: mol_type = genomic DNA
ctttaagaaggagatataccatggaagtgcagctggtggag

SEQ ID NO: 40 - DNA, 41 bp, Homo sapiens
  source 1..41: mol_type = genomic DNA
ctttaagaaggagatataccatggaggtgcagctggtggag

SEQ ID NO: 41 - DNA, 41 bp, Homo sapiens
  source 1..41: mol_type = genomic DNA
ctttaagaaggagatataccatgcaggtgcagctggtggag

SEQ ID NO: 42 - DNA, 41 bp, Homo sapiens
  source 1..41: mol_type = genomic DNA
ctttaagaaggagatataccatggaggtgcagctgttggag

SEQ ID NO: 43 - DNA, 41 bp, Homo sapiens
  source 1..41: mol_type = genomic DNA
ctttaagaaggagatataccatgcagctgcagctgcaggag

SEQ ID NO: 44 - DNA, 41 bp, Homo sapiens
  source 1..41: mol_type = genomic DNA
ctttaagaaggagatataccatgcaggtgcagctgcaggag

SEQ ID NO: 45 - DNA, 41 bp, Homo sapiens
  source 1..41: mol_type = genomic DNA
ctttaagaaggagatataccatgcaggtgcagctacagcag

SEQ ID NO: 46 - DNA, 41 bp, Homo sapiens
  source 1..41: mol_type = genomic DNA
ctttaagaaggagatataccatggaagtgcagctggtgcag

SEQ ID NO: 47 - DNA, 41 bp, Homo sapiens
  source 1..41: mol_type = genomic DNA
ctttaagaaggagatataccatggaggtgcagctggtgcag

SEQ ID NO: 48 - DNA, 41 bp, Homo sapiens
  source 1..41: mol_type = genomic DNA
ctttaagaaggagatataccatgcaggtacagctgcagcag

SEQ ID NO: 49 - DNA, 41 bp, Homo sapiens
  source 1..41: mol_type = genomic DNA
ctttaagaaggagatataccatgcaggtccagctggtgcaa

SEQ ID NO: 50 - DNA, 41 bp, Homo sapiens
  source 1..41: mol_type = genomic DNA
ctttaagaaggagatataccatgcaggtgcagctggtgcaa

SEQ ID NO: 51 - DNA, 45 bp, Homo sapiens
  source 1..45: mol_type = genomic DNA
atcgtatgggtagctggtcccttagaccgatgggcccttggtgga

SEQ ID NO: 52 - DNA, 45 bp, Homo sapiens
  source 1..45: mol_type = genomic DNA
atcgtatgggtagctggtcccttagacggatgggcccttggtgga

SEQ ID NO: 53 - DNA, 41 bp, Homo sapiens
  source 1..41: mol_type = genomic DNA
ctttaagaaggagatataccatgaacatccagatgacccag

SEQ ID NO: 54 - DNA, 41 bp, Homo sapiens
  source 1..41: mol_type = genomic DNA
ctttaagaaggagatataccatggacatccagatgacccag

SEQ ID NO: 55 - DNA, 41 bp, Homo sapiens
  source 1..41: mol_type = genomic DNA
ctttaagaaggagatataccatggacatccagttgacccag

SEQ ID NO: 56 - DNA, 41 bp, Homo sapiens
  source 1..41: mol_type = genomic DNA
ctttaagaaggagatataccatggccatccagttgacccag

SEQ ID NO: 57 - DNA, 41 bp, Homo sapiens
  source 1..41: mol_type = genomic DNA
ctttaagaaggagatataccatggccatccagatgacccag

SEQ ID NO: 58 - DNA, 41 bp, Homo sapiens
  source 1..41: mol_type = genomic DNA
ctttaagaaggagatataccatggccatccggatgacccag

SEQ ID NO: 59 - DNA, 41 bp, Homo sapiens
  source 1..41: mol_type = genomic DNA
ctttaagaaggagatataccatggtcatctggatgacccag

SEQ ID NO: 60 - DNA, 41 bp, Homo sapiens
  source 1..41: mol_type = genomic DNA
ctttaagaaggagatataccatggatattgtgatgacccag

SEQ ID NO: 61 - DNA, 41 bp, Homo sapiens
  source 1..41: mol_type = genomic DNA
ctttaagaaggagatataccatggatattgtgatgactcag

SEQ ID NO: 62 - DNA, 41 bp, Homo sapiens
  source 1..41: mol_type = genomic DNA
ctttaagaaggagatataccatggatgttgtgatgactcag

SEQ ID NO: 63 - DNA, 41 bp, Homo sapiens
  source 1..41: mol_type = genomic DNA
ctttaagaaggagatataccatggaaattgtgttgacacag

SEQ ID NO: 64 - DNA, 41 bp, Homo sapiens
  source 1..41: mol_type = genomic DNA
ctttaagaaggagatataccatggaaattgtgttgacgcag

SEQ ID NO: 65 - DNA, 41 bp, Homo sapiens
  source 1..41: mol_type = genomic DNA
ctttaagaaggagatataccatggaaatagtgatgacgcag

SEQ ID NO: 66 - DNA, 41 bp, Homo sapiens
  source 1..41: mol_type = genomic DNA
ctttaagaaggagatataccatggaaattgtaatgacacag

SEQ ID NO: 67 - DNA, 41 bp, Homo sapiens
  source 1..41: mol_type = genomic DNA
ctttaagaaggagatataccatggacatcgtgatgacccag

SEQ ID NO: 68 - DNA, 41 bp, Homo sapiens
  source 1..41: mol_type = genomic DNA
ctttaagaaggagatataccatggaaacgacactcacgcag

SEQ ID NO: 69 - DNA, 41 bp, Homo sapiens
  source 1..41: mol_type = genomic DNA
ctttaagaaggagatataccatggaaattgtgctcactcag

SEQ ID NO: 70 - DNA, 41 bp, Homo sapiens
  source 1..41: mol_type = genomic DNA
ctttaagaaggagatataccatggatgttgtgatgacacag

SEQ ID NO: 71 - DNA, 44 bp, Homo sapiens
  source 1..44: mol_type = genomic DNA
atcgtatgggtagctggtcccttaaagatgaagacagatggtgc

SEQ ID NO: 72 - DNA, 41 bp, Homo sapiens
  source 1..41: mol_type = genomic DNA
ctttaagaaggagatataccatgcagtctgtgctgactcag

SEQ ID NO: 73 - DNA, 41 bp, Homo sapiens
  source 1..41: mol_type = genomic DNA
ctttaagaaggagatataccatgcagtctgtgctgacgcag

SEQ ID NO: 74 - DNA, 41 bp, Homo sapiens
  source 1..41: mol_type = genomic DNA
ctttaagaaggagatataccatgcagtctgtgttgacgcag

SEQ ID NO: 75 - DNA, 41 bp, Homo sapiens
  source 1..41: mol_type = genomic DNA
ctttaagaaggagatataccatgcagtctgtcgtgacgcag

SEQ ID NO: 76 - DNA, 41 bp, Homo sapiens
  source 1..41: mol_type = genomic DNA
ctttaagaaggagatataccatgcagtctgccctgactcag

SEQ ID NO: 77 - DNA, 41 bp, Homo sapiens
  source 1..41: mol_type = genomic DNA
ctttaagaaggagatataccatgtcctatgagctgactcag

SEQ ID NO: 78 - DNA, 41 bp, Homo sapiens
  source 1..41: mol_type = genomic DNA
ctttaagaaggagatataccatgtcctatgtgctgactcag

SEQ ID NO: 79 - DNA, 41 bp, Homo sapiens
  source 1..41: mol_type = genomic DNA
ctttaagaaggagatataccatgtcctatgagctgacacag

SEQ ID NO: 80 - DNA, 41 bp, Homo sapiens
  source 1..41: mol_type = genomic DNA
ctttaagaaggagatataccatgtcttctgagctgactcag

SEQ ID NO: 81 - DNA, 41 bp, Homo sapiens
  source 1..41: mol_type = genomic DNA
ctttaagaaggagatataccatgtcctatgagctgatgcag

SEQ ID NO: 82 - DNA, 41 bp, Homo sapiens
  source 1..41: mol_type = genomic DNA
ctttaagaaggagatataccatgcagcctgtgctgactcaa

SEQ ID NO: 83 - DNA, 41 bp, Homo sapiens
  source 1..41: mol_type = genomic DNA
ctttaagaaggagatataccatgcagcttgtgctgactcaa

SEQ ID NO: 84 - DNA, 41 bp, Homo sapiens
  source 1..41: mol_type = genomic DNA
ctttaagaaggagatataccatgcagcctgtgctgactcag

SEQ ID NO: 85 - DNA, 41 bp, Homo sapiens
  source 1..41: mol_type = genomic DNA
ctttaagaaggagatataccatgcaggctgtgctgactcag

SEQ ID NO: 86 - DNA, 41 bp, Homo sapiens
  source 1..41: mol_type = genomic DNA
ctttaagaaggagatataccatgaattttatgctgactcag

SEQ ID NO: 87 - DNA, 41 bp, Homo sapiens
  source 1..41: mol_type = genomic DNA
ctttaagaaggagatataccatgcagactgtggtgactcag

SEQ ID NO: 88 - DNA, 41 bp, Homo sapiens
  source 1..41: mol_type = genomic DNA
ctttaagaaggagatataccatgcaggctgtggtgactcag

SEQ ID NO: 89 - DNA, 41 bp, Homo sapiens
  source 1..41: mol_type = genomic DNA
ctttaagaaggagatataccatgcagactgtggtgacccag

SEQ ID NO: 90 - DNA, 41 bp, Homo sapiens; identical to SEQ ID NO: 84
  source 1..41: mol_type = genomic DNA

SEQ ID NO: 91 - DNA, 41 bp, Homo sapiens
  source 1..41: mol_type = genomic DNA
ctttaagaaggagatataccatgctgcctgtgctgactcag

SEQ ID NO: 92 - DNA, 41 bp, Homo sapiens
  source 1..41: mol_type = genomic DNA
ctttaagaaggagatataccatgcaggcagggctgactcag

SEQ ID NO: 93 - DNA, 40 bp, Homo sapiens
  source 1..40: mol_type = genomic DNA
atcgtatgggtagctggtcccttagggaacagagtgaccg

SEQ ID NO: 94 - DNA, 43 bp, Homo sapiens
  source 1..43: mol_type = genomic DNA
ctttaagaaggagatataccatgcaggtgcagctggtgcagtc

SEQ ID NO: 95 - DNA, 46 bp, Homo sapiens
  source 1..46: mol_type = genomic DNA
ctttaagaaggagatataccatgcaggtcaacttaagggagtctgg

SEQ ID NO: 96 - DNA, 42 bp, Homo sapiens
  source 1..42: mol_type = genomic DNA
ctttaagaaggagatataccatgaggtgcagctgctgcagtc

SEQ ID NO: 97 - DNA, 42 bp, Homo sapiens
  source 1..42: mol_type = genomic DNA
ctttaagaaggagatataccatgaggtgcagctgctggagtc

SEQ ID NO: 98 - DNA, 42 bp, Homo sapiens
  source 1..42: mol_type = genomic DNA
ctttaagaaggagatataccatgaggtgcagctggtgcagtc

SEQ ID NO: 99 - DNA, 42 bp, Homo sapiens
  source 1..42: mol_type = genomic DNA
ctttaagaaggagatataccatgaggtgcagctggtggagtc

SEQ ID NO: 100 - DNA, 43 bp, Homo sapiens
  source 1..43: mol_type = genomic DNA
ctttaagaaggagatataccatgcaggtacagctgcagcagtc

SEQ ID NO: 101 - DNA, 43 bp, Homo sapiens
  source 1..43: mol_type = genomic DNA
ctttaagaaggagatataccatgcaggtacagctgcaggagtc

SEQ ID NO: 102 - DNA, 43 bp, Homo sapiens
  source 1..43: mol_type = genomic DNA
ctttaagaaggagatataccatgcaggtgcagctgcagcagtc

SEQ ID NO: 103 - DNA, 43 bp, Homo sapiens
  source 1..43: mol_type = genomic DNA
ctttaagaaggagatataccatgcaggtgcagctgcaggagtc

SEQ ID NO: 104 - DNA, 67 bp, Homo sapiens
  source 1..67: mol_type = genomic DNA
atcgtatgggtagctggtcccttagtggtggtggtggtggtgaactctcttgtccaccttggtgttg

SEQ ID NO: 105 - DNA, 67 bp, Homo sapiens
  source 1..67: mol_type = genomic DNA
atcgtatgggtagctggtcccttagtggtggtggtggtggtgaactgtcttgtccaccttggtgttg

SEQ ID NO: 106 - DNA, 67 bp, Homo sapiens
  source 1..67: mol_type = genomic DNA
atcgtatgggtagctggtcccttagtggtggtggtggtggtgaactttcttgtccaccttggtgttg

SEQ ID NO: 107 - DNA, 44 bp, Homo sapiens
  source 1..44: mol_type = genomic DNA
ctttaagaaggagatataccatggacatccagatgacccagtct

SEQ ID NO: 108 - DNA, 44 bp, Homo sapiens
  source 1..44: mol_type = genomic DNA
ctttaagaaggagatataccatggacatcgagatgacccagtct

SEQ ID NO: 109 - DNA, 44 bp, Homo sapiens; identical to SEQ ID NO: 108
  source 1..44: mol_type = genomic DNA

SEQ ID NO: 110 - DNA, 44 bp, Homo sapiens
  source 1..44: mol_type = genomic DNA
ctttaagaaggagatataccatggacatcgtgatgacccagtct

SEQ ID NO: 111 - DNA, 46 bp, Homo sapiens
  source 1..46: mol_type = genomic DNA
ctttaagaaggagatataccatggatattgtgatgactcagtctcc

SEQ ID NO: 112 - DNA, 46 bp, Homo sapiens
  source 1..46: mol_type = genomic DNA
ctttaagaaggagatataccatggatattgtgctgactcagtctcc

SEQ ID NO: 113 - DNA, 46 bp, Homo sapiens
  source 1..46: mol_type = genomic DNA
ctttaagaaggagatataccatggaaattgtgttgacgcagtctcc

SEQ ID NO: 114 - DNA, 45 bp, Homo sapiens
  source 1..45: mol_type = genomic DNA
ctttaagaaggagatataccatggaaacgacactcacgcagtctc

SEQ ID NO: 115 - DNA, 46 bp, Homo sapiens
  source 1..46: mol_type = genomic DNA
atcgtatgggtagctggtcccttaacactctcccctgttgaagctc

SEQ ID NO: 116 - DNA, 43 bp, Homo sapiens
  source 1..43: mol_type = genomic DNA
ctttaagaaggagatataccatgcagtctgtgctgactcagcc

SEQ ID NO: 117 - DNA, 43 bp, Homo sapiens
  source 1..43: mol_type = genomic DNA
ctttaagaaggagatataccatgcagtctgccctgactcagcc

SEQ ID NO: 118 - DNA, 43 bp, Homo sapiens
  source 1..43: mol_type = genomic DNA
ctttaagaaggagatataccatgtcctatgagctgacacagcc

SEQ ID NO: 119 - DNA, 43 bp, Homo sapiens
  source 1..43: mol_type = genomic DNA
ctttaagaaggagatataccatgtcctatgagctgactcagcc

SEQ ID NO: 120 - DNA, 46 bp, Homo sapiens
  source 1..46: mol_type = genomic DNA
atcgtatgggtagctggtcccttatgaacattccgcaggggcaact

SEQ ID NO: 121 - DNA, 46 bp, Homo sapiens
  source 1..46: mol_type = genomic DNA
atcgtatgggtagctggtcccttatgaacattctgcaggggcaact

SEQ ID NO: 122 - DNA, 46 bp, Homo sapiens
  source 1..46: mol_type = genomic DNA
atcgtatgggtagctggtcccttatgaacattccgtaggggcaact

SEQ ID NO: 123 - DNA, 46 bp, Homo sapiens
  source 1..46: mol_type = genomic DNA
atcgtatgggtagctggtcccttatgaacattccgcaggggctact

SEQ ID NO: 124 - DNA, 46 bp, Homo sapiens
  source 1..46: mol_type = genomic DNA
atcgtatgggtagctggtcccttatgaacattctgtaggggcaact

SEQ ID NO: 125 - DNA, 46 bp, Homo sapiens
  source 1..46: mol_type = genomic DNA
atcgtatgggtagctggtcccttatgaacattctgtaggggctact

SEQ ID NO: 126 - DNA, 23 bp, Homo sapiens
  source 1..23: mol_type = genomic DNA
ctttaagaaggagatataccatg

SEQ ID NO: 127 - DNA, 24 bp, Homo sapiens
  source 1..24: mol_type = genomic DNA
atcgtatgggtagctggtccctta

SEQ ID NO: 128 - DNA, 41 bp, Homo sapiens
  source 1..41: mol_type = genomic DNA
ctttaagaaggagatataccatgcaggtkcagctggtgcag

SEQ ID NO: 129 - DNA, 41 bp, Homo sapiens; identical to SEQ ID NO: 31
  source 1..41: mol_type = genomic DNA

SEQ ID NO: 130 - DNA, 41 bp, Homo sapiens
  source 1..41: mol_type = genomic DNA
ctttaagaaggagatataccatgsaggtccagctggtacag

SEQ ID NO: 131 - DNA, 41 bp, Homo sapiens
  source 1..41: mol_type = genomic DNA
ctttaagaaggagatataccatgcaratgcagctggtgcag

SEQ ID NO: 132 - DNA, 41 bp, Homo sapiens; identical to SEQ ID NO: 36
  source 1..41: mol_type = genomic DNA

SEQ ID NO: 133 - DNA, 41 bp, Homo sapiens
  source 1..41: mol_type = genomic DNA
ctttaagaaggagatataccatgcaggtcaccttgarggag

SEQ ID NO: 134 - DNA, 41 bp, Homo sapiens
  source 1..41: mol_type = genomic DNA
ctttaagaaggagatataccatggargtgcagctggtggag

SEQ ID NO: 135 - DNA, 41 bp, Homo sapiens; identical to SEQ ID NO: 41
  source 1..41: mol_type = genomic DNA

SEQ ID NO: 136 - DNA, 41 bp, Homo sapiens; identical to SEQ ID NO: 42
  source 1..41: mol_type = genomic DNA

SEQ ID NO: 137 - DNA, 41 bp, Homo sapiens
  source 1..41: mol_type = genomic DNA
ctttaagaaggagatataccatgcagstgcagctgcaggag

SEQ ID NO: 138 - DNA, 41 bp, Homo sapiens; identical to SEQ ID NO: 45
  source 1..41: mol_type = genomic DNA

SEQ ID NO: 139 - DNA, 41 bp, Homo sapiens
  source 1..41: mol_type = genomic DNA
ctttaagaaggagatataccatggargtgcagctggtgcag

SEQ ID NO: 140 - DNA, 41 bp, Homo sapiens; identical to SEQ ID NO: 48
  source 1..41: mol_type = genomic DNA

SEQ ID NO: 141 - DNA, 41 bp, Homo sapiens
  source 1..41: mol_type = genomic DNA
ctttaagaaggagatataccatgcaggtacagctggtgcaa

SEQ ID NO: 142 - DNA, 48 bp, synthetic construct
  misc_feature 1..48: note = VH-lfp
  source 1..48: mol_type = other DNA
ctttaagaaggagatataccatgaactbtcttgtccaccttggtgttg

SEQ ID NO: 143 - DNA, 49 bp, synthetic construct
  misc_feature 1..49: note = VH-rfp
  source 1..49: mol_type = other DNA
atcgtatgggtagctggtcccttaaactbtcttgtccaccttggtgttg

SEQ ID NO: 144 - DNA, 45 bp, synthetic construct
  misc_feature 1..45: note = VL(k)-lfp
  source 1..45: mol_type = other DNA
ctttaagaaggagatataccatgacactctcccctgttgaagctc

SEQ ID NO: 145 - DNA, 46 bp, synthetic construct; identical to SEQ ID NO: 115
  misc_feature 1..46: note = VL(k)-rfp
  source 1..46: mol_type = other DNA

SEQ ID NO: 146 - protein, 4 aa, synthetic construct
  REGION 1..4: note = Sythetic Construct
ELLG

SEQ ID NO: 147 - protein, 4 aa, synthetic construct
  REGION 1..4: note = Synthetic Construct
EFLG

(19) United States
(12) Patent Application Publication
(10) Pub. No.: US 2025/0137028 A1
(43) Pub. Date: May 1, 2025

(54) METHOD FOR OBTAINING IMMUNOGLOBULIN ENCODING NUCLEIC ACID

(71) Applicant: Hoffmann-La Roche Inc., Little Falls, NJ (US)

(72) Inventors: Hans-Willi Krell, Penzberg (DE); Alexander Lifke, Penzberg (DE); Valeria Lifke, Penzberg (DE); Kairat Madin, Penzberg (DE); Christian Weilke, Penzberg (CH)

(73) Assignee: Hoffmann-La Roche Inc., Little Falls, NJ (US)

(21) Appl. No.: 18/762,599

(22) Filed: Jul. 2, 2024

Related U.S. Application Data
(63) Continuation of application No. 15/188,870, filed on Jun. 21, 2016, now abandoned.
Application No. 15/188,870 is a division of application No. 13/202,446, filed on Aug. 19, 2011, now Pat. No. 9,399,670.

(30) Foreign Application Priority Data
Feb. 20, 2009 (EP) 09002396.1

(51) Int. Cl. C12P 21/00; C07K 16/00; C12P 19/34; C12Q 1/6804; C12Q 1/6858; C12Q 1/6881

(52) U.S. Cl. CPC C12P 21/005; C07K 16/00; C12P 19/34; C12Q 1/6804; C12Q 1/6858; C12Q 1/6881; C07K 2317/14; C07K 2317/21; C07K 2317/55; C12Q 2537/143; C12Q 2549/119; C12Q 2600/16

(57) ABSTRACT

The current invention is directed to a method for obtaining a nucleic acid encoding an immunoglobulin variable domain from a single cell comprising the following steps:
Performing a first polymerase chain reaction with three to six 5′-primers and one 3′-primer,
Performing with the product of the first polymerase chain reaction a second polymerase chain reaction with thirteen to sixteen primers and one 3′-primer,

whereby the distance of the binding locations of the primer employed in the second polymerase chain reaction is reduced compared to the first polymerase chain reaction.

### CROSS REFERENCE TO RELATED APPLICATIONS

This application is a continuation of U.S. application Ser. No. 15/188,870 filed Jun. 21, 2016 which is a divisional of U.S. application Ser. No. 13/202,446, filed Aug. 19, 2011, now U.S. Pat. No. 9,399,670, which is a national stage application of International Application No. PCT/EP2010/001008 having an international filing date of Feb. 18, 2010 and which claims benefit under 35 U.S.C. § 119 to European Patent Application No. 09002396.1, filed Feb. 20, 2009, the contents of which are hereby incorporated by reference.

### SEQUENCE LISTING

The instant application contains a Sequence Listing which has been submitted electronically in XML format and is hereby incorporated by reference in its entirety. Said XML copy, created Jul. 1, 2024 is named P25241-US-2_Seq_List.xml and is 135,477 bytes in size.

### FIELD OF THE INVENTION

The present invention relates to a method and means for obtaining immunoglobulin encoding nucleic acid from a single immunoglobulin producing cell with a multiplexed polymerase chain reaction (PCR), and also to a method for producing an immunoglobulin whereby the immunoglobulin encoding nucleic acid is obtained from a single immunoglobulin producing cell in combination with in vitro translation. Also encompassed by the current invention is a method for characterization of recombinantly produced human Fab-fragments.

### BACKGROUND OF THE INVENTION

Since the establishment of hybridoma technology (Cole, S. P. C., et al., Monoclonal Antibodies and Cancer Therapy, Alan R. Liss, p. 77 (1985); and Boerner, P., et al., J. Immunol. 147 (1991) 86-95), monoclonal immunoglobulins have emerged to play a pivotal role in scientific research, human healthcare and diagnostics. Consequently, the generation of monoclonal, especially therapeutic, immunoglobulins is a field undergoing intensive research. In this respect, the hybridoma technology and phage display technology (Hoogenboom, H. R., and Winter, G., J. Mol. Biol. 227 (1992) 381-388; Marks, J. D., et al., J. Mol. Biol. 222 (1991) 581-597) are, amongst others, two commonly used technologies for the generation of monoclonal immunoglobulins. In hybridoma technology obtaining of stable clones is a hurdle, thus, diminishing diversity of the antibodies, as only a limited number of B-cells are successfully fused, propagated and thereafter characterized. Similarly, a drawback of phage or yeast display-based combinatorial library approaches is the random pairing of the immunoglobulin heavy and light chains. The dissociation of the original heavy and light chain pairing, and non-cognate pairing, necessitate the screening of a large number of immunoglobulin producing cells in order to identify heavy and light chain pairs of high affinity. In addition, such non-cognate pairs may display unwanted cross-reactivity to human antigens. Finally, the genetic diversity of target-specific immunoglobulins identified by selection and screening of combinatorial libraries is commonly limited due to inherent selection biases.

Generation of immunoglobulins from immunoglobulin producing cell can be performed according to methods known in the art. Such methods are e.g. hybridoma technique. A different method is based on the identification of the nucleic acid sequence of the immunoglobulin. Usually it is sufficient to identify the sequence of the variable regions or even only the CDR regions or only the CDR3 region. For example, the mRNA is isolated from a pool of immunoglobulin producing cells and is used for the construction of a cDNA-library encoding the CDR regions of the immunoglobulin. The cDNA-library is then transfected into a suitable host cell, such as NS0 or CHO, and screened for specific immunoglobulin production.

WO 2008/104184 reports a method for cloning cognate antibodies. The efficient generation of monoclonal antibodies from single human B cells is reported by Tiller et al. (Tiller, T., et al., J. Immunol. Meth. 329 (2007) 112-124). Braeuninger et al. (Braeuninger, A., et al., Blood 93 (1999) 2679-2687) report the molecular analysis of single B cells from T-cell-rich B-cell lymphoma. Systematic design and testing of nested (RT-) PCR primer is reported by Rohatgi et al. (Rohatgi, S., et al, J. Immunol. Meth. 339 (2008) 205-219). In WO 02/13862 a method and composition for altering a B-cell mediated pathology are reported. Haurum et al. (Meijer, P. J. and Haurum, J. S., J. Mol. Biol. 358 (2006) 764-772) report a one-step RT-multiplex overlap extension PCR. Stollar et al. and Junghans et al. report the sequence analysis by single cell PCR reaction (Wang, X. and Stollar, B. D., J. Immunol. Meth. 244 (2000) 217-225; Coronella, J. A. and Junghans, R. P., Nucl. Acids Res. 28 (2000) E85). Jiang, X. and Nakano, H., et al. (Biotechnol. Prog. 22 (2006) 979-988) report the construction of a linear expression element for in vitro transcription and translation.

### SUMMARY OF THE INVENTION

The current invention is directed in specific embodiments of an aspect to a method for providing a human monoclonal antibody comprising the in vitro translation of a nucleic acid encoding human immunoglobulin G fragments whereby the nucleic acid is obtained by specific amplification of cDNA fragments obtained from the mRNA of a single immunoglobulin producing human B-cell, plasmablast or plasma cell or a B-cell of an animal comprising a human immunoglobulin locus.

With this method it is possible to characterize each of a number of provided B-cells with respect to the antigen binding characteristics of the produced immunoglobulin. Thus, no loss of immunoglobulin diversity occurs. As the analyzed B-cells are mature B-cells obtained after the in vivo maturation process it is very unlikely that their produced immunoglobulins show cross-reactivity with other antigens.

The invention comprises a method for the multiplex semi-nested PCR and multiplex one tube RT-GSP-PCR (RT-Gene Specific Primer-PCR) for the amplification of cognate IgG HC and IgG LC chains (human IgG isotype) from a single B-cell or plasmablast or plasma cell. The Fab PCR product is subsequently transcribed to mRNA and translated in vitro in E. coli lysate. The expression was examined using ELISA and Western blot.

The current invention comprises as first aspect a method for obtaining a nucleic acid encoding an immunoglobulin variable domain from a single cell comprising the following steps:
obtaining a first nucleic acid composition by performing a first polymerase chain reaction with three to six 5′-primer and one 3′-primer,
obtaining a nucleic acid encoding an immunoglobulin variable domain by performing with the composition obtained in the first polymerase chain reaction a second polymerase chain reaction with thirteen to sixteen 5′-primer and one 3′-primer,
whereby, when bound to the nucleic acid to be amplified, the distance of the binding locations of the 5′-primer to the binding location of the 3′-primer employed in the second polymerase chain reaction is reduced compared to that of the first polymerase chain reaction.

A second aspect of the current invention is a method for obtaining a nucleic acid encoding an immunoglobulin variable domain from a single cell comprising the following steps:
obtaining a nucleic acid composition by performing a first polymerase chain reaction with four to six 5′-primer and one 3′-primer,
obtaining a nucleic acid encoding an immunoglobulin variable domain by performing with the composition obtained in the first polymerase chain reaction a second polymerase chain reaction with thirteen to fifteen 5′-primer and one 3′-primer,
whereby in the second polymerase chain reaction either the 5′-primer are the same as that in the first polymerase chain reaction and the 3′-primer is changed or the 3′-primer is the same as in the first polymerase chain reaction and at least one 5′-primer is changed,
whereby, when bound to the nucleic acid to be amplified, the number of nucleotides between the 5′-end of each of the 5′-primer and the 3′-end of the 3′-primer in the second polymerase chain reaction is reduced compared to the number of nucleotides between the 5′-end of each of the 5′-primer and the 3′-end of the 3′-primer in the first polymerase chain reaction.

A further aspect of the current invention is a method for obtaining a nucleic acid encoding an immunoglobulin variable domain from a single cell by a multiplex one tube RT-GSP-PCR comprising the following step:
performing a reverse transcription and polymerase chain reaction in one step with one 5′-primer and one 3′-primer.

In one embodiment the methods according to the invention are characterized in that the 5′-primer employed in the first polymerase chain reaction bind in the coding region for the leader peptide of the immunoglobulin. In another embodiment the methods according to the invention are characterized in that the 5′-primer employed in the second polymerase chain reaction or the 5′-primer employed in a multiplex one tube RT-GSP-PCR bind in the coding region of the first framework region of the immunoglobulin. In a further embodiment the methods according to the invention are characterized in that the primer employed in the second polymerase chain reaction provide for overhangs encoding the translational start codon ATG for 5′-primer and/or the translational stop codon TTA for 3′-primer. In still a further embodiment the methods according to the invention are characterized in comprising the additional step of:
providing a single cell and obtaining the mRNA of this cell.

In a further embodiment the methods according to the previous embodiment are characterized in comprising the following second step:
obtaining cDNA from the provided mRNA with a reverse transcriptase polymerase chain reaction (RT-PCR).

In another embodiment the methods according to the invention are characterized in that six 5′-primer and one 3′-primer are employed in the first polymerase chain reaction. In still a further embodiment the methods according to the invention are characterized in that four 5′-primer and one 3′-primer are employed in the second polymerase chain reaction. In a further embodiment of the current invention the methods are characterized in that
a) for obtaining a nucleic acid encoding an immunoglobulin heavy chain variable domain the primer in the first polymerase chain reaction comprise the nucleic acids of SEQ ID NO: 05 and/or 06, 07 and/or 08, 09, 10 and/or 11, 12, 13, and 104 and/or 105 and/or 106, and the primer in the second polymerase chain reaction comprise the nucleic acids of SEQ ID NO: 128, 129, 130, 131, 132, 133, 134, 135, 136, 137, 138, 139, 140, 141, and 104 and/or 105 and/or 106, and/or 142, and/or 143,
b) for obtaining a nucleic acid encoding an immunoglobulin kappa light chain variable domain the primer in the first polymerase chain reaction comprise the nucleic acids of SEQ ID NO: 16, 17, 18, 19, and 115, and the primer in the second polymerase chain reaction comprise the nucleic acids of SEQ ID NO: 53 and/or 54, 55 and/or 56, 57 and/or 58, 59, 60, 61 and/or 62, 63 and/or 64, 65, 66, 67, 68, 69, 70, and/or 115, and/or 144, and/or 145,
c) for obtaining a nucleic acid encoding an immunoglobulin lambda light chain variable domain the primer in the first polymerase chain reaction comprise the nucleic acids of SEQ ID NO: 21, 22, 23 and/or 24 and/or 25 and/or 26, and 120and/or 121 and/or 122 and/or 123 and/or 124 and/or 125, and the primer in the second polymerase chain reaction comprise the nucleic acids of SEQ ID NO: 72, 73 and/or 74, 75, 76, 77 and/or 78, 79, 80, 81, 82 and/or 83, 84 and/or 85, 86, 87 and/or 88, 89, 90 and/or 91, 92, and 120 and/or 121 and/or 122 and/or 123 and/or 124 and/or 125.

In one embodiment of the methods according to the invention the immunoglobulin variable domain is an immunoglobulin heavy chain variable domain or an immunoglobulin kappa light chain variable domain or an immunoglobulin lambda light chain variable domain.

A further aspect of the current invention is a method for producing an immunoglobulin Fab-fragment comprising the following steps:
providing a single immunoglobulin producing cell,
obtaining from the cell the nucleic acid encoding the immunoglobulin light and heavy chain variable domains, optionally also encoding a part of the light chain constant domain and a part of the heavy chain CHI domain,
generating a linear expression matrix comprising the obtained nucleic acid,
translating in vitro the nucleic acid and thereby producing the immunoglobulin Fab fragment.

Another aspect of the current invention is a method for producing an immunoglobulin comprising the following steps:
providing a single immunoglobulin producing cell,
obtaining from the cell the nucleic acid encoding the immunoglobulin light and heavy chain variable domains,
linking the nucleic acid encoding the light chain variable domain with a nucleic acid encoding an immunoglobulin light chain constant domain in operable form, and linking the nucleic acid encoding the heavy chain variable domain with a nucleic acid encoding an immunoglobulin heavy chain constant region in operable form,
transfecting a eukaryotic or a prokaryotic cell with the nucleic acids obtained in the previous step,
cultivating the transfected cell, in one embodiment under conditions suitable for the expression of the immunoglobulin,
recovering the immunoglobulin from the cell or the cultivation medium and thereby producing an immunoglobulin.

In one embodiment of all methods according to the invention is the immunoglobulin an immunoglobulin of class G (IgG). In one embodiment of the methods for producing an immunoglobulin Fab fragment or an immunoglobulin is the obtaining of the nucleic acid by a method according to an aspect of the current invention.

### DESCRIPTION OF THE INVENTION

One aspect of the current invention is a method for obtaining a nucleic acid encoding an immunoglobulin variable domain from a single cell comprising the following steps:
performing a first polymerase chain reaction with three to six 5′-primer and one 3′-primer,
performing with the product of the first polymerase chain reaction a second polymerase chain reaction with thirteen to sixteen 5′-primer and one 3′-primer, thereby obtaining a nucleic acid encoding an immunoglobulin variable domain,
whereby the distance of the binding locations of the primer employed in the second polymerase chain reaction is reduced compared to the first polymerase chain reaction.

By employing magnetic micro-beads coated with the human pan B-cell marker, CD19 (see e.g. Bertrand, F. E., III, et al., Blood 90 (1997) 736-744), B-cells were isolated from peripheral blood. With the limited dilution approach, single cells were placed in a 96 well microtiter plate. The mRNA of these cells was extracted.

It has been found that by performing the IgG-specific PCR amplification according to the current invention for obtaining nucleic acid encoding an immunoglobulin variable domain from a single cell in a thereafter following production of the respective immunoglobulin or Fab-fragment in one embodiment OD-values in the range of from 0.5 to 2.0 were obtained, and concomitantly, e.g., Fab-fragment yields of from 180 to 310 ng/ml were obtained.

With the methods according to the current invention a multiplex polymerase chain reaction is used for the amplification of heavy and light chain variable domains simultaneously in the same polymerase chain reaction. In contrast to the amplification of the heavy chain variable domain and the light chain variable domain in separate reactions the current approach provides for an increased sensitivity and an increased amount of amplified sequences. The use of gene-specific primer in both, i.e. all, polymerase chain reactions enhances the specificity and accuracy of the methods.

More complex gene structure in the case of human IgG requires a different strategy for the primer design, placement and polymerase chain reaction for the sensitivity and accuracy required.

Thus, herein is employed a multiplex polymerase chain reaction either without or with the linkage of the heavy and light chain regions that are amplified. For the in vitro translation of the obtained nucleic acids it is beneficial that the encoded domains comprise cysteine residues suitable for the formation of interchain disulfide bonds.

Methods and techniques known to a person skilled in the art, which are useful for carrying out the current invention, are described e.g. in Ausubel, F. M., ed., Current Protocols in Molecular Biology, Volumes I to III (1997), Wiley and Sons; Sambrook, J., et al., Molecular Cloning: A Laboratory Manual, Second Edition, Cold Spring Harbor Laboratory Press, Cold Spring Harbor, N.Y. (1989), Morrison, S.L., et al., Proc. Natl. Acad. Sci. USA 81 (1984) 6851-6855; U.S. Pat. Nos. 5,202,238 and 5,204,244.

The term “immunoglobulin” denotes a protein consisting of one or more polypeptide(s) substantially encoded by immunoglobulin genes. The recognized immunoglobulin genes include the different constant region genes as well as the myriad immunoglobulin variable region genes. Immunoglobulins may exist in a variety of formats, including, for example, Fv, Fab, and F(ab)₂ as well as single chains (scFv) or diabodies (e.g. Huston, J. S., et al., Proc. Natl. Acad. Sci. USA 85 (1988) 5879-5883; Bird, R. E., et al., Science 242 (1988) 423-426; Hood, L. E. et al., Immunology, Benjamin N.Y., 2nd edition (1984); Hunkapiller, T. and Hood, L., Nature 323 (1986) 15-16).

An immunoglobulin in general comprises two so called light chain polypeptides (light chain) and two so called heavy chain polypeptides (heavy chain). Each of the heavy and light chain polypeptides contains a variable domain (variable region) (generally the amino terminal portion of the polypeptide chain) comprising binding regions that are able to interact with an antigen. Each of the heavy and light chain polypeptides comprises a constant region (generally the carboxyl terminal portion). The constant region of the heavy chain mediates the binding of the antibody i) to cells bearing a Fc gamma receptor (FcγR), such as phagocytic cells, or ii) to cells bearing the neonatal Fc receptor (FcRn) also known as Brambell receptor. It also mediates the binding to some factors including factors of the classical complement system such as component (Clq). The variable domain of an immunoglobulin's light or heavy chain in turn comprises different segments, i.e. four framework regions (FR) and three hypervariable regions (CDR).

Genetic engineering of immunoglobulins is e.g. described in Morrison, S. L., et al., Proc. Natl. Acad Sci. USA 81 (1984) 6851-6855; U.S. Pat. Nos. 5,202,238 and 5,204,244; Riechmann, L., et al., Nature 332 (1988) 323-327; Neuberger, M. S., et al., Nature 314 (1985) 268-270; Lonberg, N., Nat. Biotechnol. 23 (2005) 1117-1125.

The term “chimeric immunoglobulin” denotes an immunoglobulin, preferably a monoclonal immunoglobulin, comprising a variable domain, i.e. binding region, from a first non-human species and at least a portion of a constant region derived from a second different source or species. Chimeric immunoglobulins are generally prepared by recombinant DNA techniques. In one embodiment chimeric immunoglobulins comprise a mouse, rat, hamster, rabbit, or sheep variable domain and a human constant region. In one embodiment the human heavy chain constant region is a human IgG constant region. In another embodiment the human light chain constant region is a kappa chain or a lambda chain.

Other forms of chimeric immunoglobulins encompassed by the present invention are those in which the class or subclass of the non-human immunoglobulin from which the variable domain is derived has been changed. Such immunoglobulins are also referred to as “class-switched immunoglobulins”. Forms of “class-switched immunoglobulins” encompassed by the present invention are also those in which the constant region has differences from the wild-type constant region sequence that result in an immunoglobulin with different properties, e.g. in regard to Clq binding and/or Fc receptor (FcR) binding. The “Fc part” of an immunoglobulin is not directly involved in binding to the antigen, but exhibit various effector functions. Depending on the amino acid sequence of the constant region of the heavy chain, immunoglobulins are divided in the classes: IgA, IgD, IgE, IgG, and IgM. Some of these classes are further divided into subclasses, i.e. IgG in IgG1, IgG2, IgG3, and IgG4, or IgA in IgA1 and IgA2. According to the immunoglobulin class to which an immunoglobulin belongs the heavy chain constant regions of immunoglobulins are called α (IgA), δ (IgD), ϵ (IgE), γ (IgG), and μ (IgM), □respectively. The immunoglobulins according to the invention belong in one embodiment to the IgG class. An “Fc part of an immunoglobulin” is a term well known to the skilled artisan and defined on basis of the papain cleavage of immunoglobulins. In one embodiment of the invention the immunoglobulin contains as Fc part a human Fc part or an Fc part derived from human origin. In a further embodiment of the invention is the Fc part either an Fc part of a human immunoglobulin of the subclass IgG4 or IgG1 or is an Fc part of a human antibody of the subclass IgG1, IgG2, or IgG3, which is modified in such a way that no Fcγ receptor (e.g. FcγRIIIa) binding and/or no Clq binding as defined below can be detected. In one embodiment the Fc part is a human Fc part, in another embodiment a human IgG4 or IgG1 subclass Fc part or a mutated

Fc part from human IgG1 subclass. In a further embodiment the Fc part is from human IgG1 subclass with mutations L234A and L235A. While IgG4 shows reduced Fcγ receptor (FcγRIIIa) binding, immunoglobulins of other IgG subclasses show strong binding. However Pro238, Asp265, Asp270, Asn297 (loss of Fc carbohydrate), Pro329, Leu234, Leu235, Gly236, Gly237, Ile253, Ser254, Lys288, Thr307, Gln311, Asn434, or/and His435 are residues which, if altered, provide also reduced FCγ receptor binding (Shields, R. L., et al., J. Biol. Chem. 276 (2001) 6591-6604; Lund, J., et al., FASEB J. 9 (1995) 115-119; Morgan, A., et al., Immunology 86 (1995) 319-324; EP 0 307 434). In one embodiment the immunoglobulin is in regard to FCγ receptor binding of IgG4 or IgG1 subclass or of IgG1 or IgG2 subclass, with a mutation in L234, L235, and/or D265, and/or contains the PVA236 mutation. In another embodiment the mutations are S228P, L234A, L235A, L235E, and/or PVA236 (PVA236 means that the amino acid sequence ELLG (given in one letter amino acid code) from amino acid position 233 to 236 of IgG1 or EFLG of IgG4 is replaced by PVA). In a further embodiment the mutations are S228P of IgG4, and L234A and L235A of IgG1. The Fc part of an immunoglobulin is directly involved in ADCC (antibody-dependent cell-mediated cytotoxicity) and CDC (complement-dependent cytotoxicity). An immunoglobulin which does not bind FCγ receptor and/or complement factor Clq does not elicit antibody-dependent cellular cytotoxicity (ADCC) and/or complement dependent cytotoxicity (CDC). In one embodiment the heavy chain constant region has an amino acid sequences of SEQ ID NO: 01, or SEQ ID NO: 02, or SEQ ID NO: 01 with mutations L234A and L235A, or SEQ ID NO: 02 with mutation S228P, and the light chain constant region has an amino acid sequence of SEQ ID NO: 03 or SEQ ID NO: 04.

“Humanized” or “CDR-grafted” forms of non-human (e.g. rodent or rabbit) immunoglobulins are immunoglobulins that contain partial sequences derived from a non-human immunoglobulin and partial sequences derived from a human immunoglobulin. For the most part, humanized immunoglobulins are derived from a human immunoglobulin (recipient or acceptor immunoglobulin), in which residues from a hypervariable region are replaced by residues from a hypervariable region of a non-human species (donor immunoglobulin), such as mouse, rat, hamster, rabbit, or non-human primate, having the desired specificity and affinity (see e.g. Morrison, S. L., et al., Proc. Natl. Acad. Sci. USA 81 (1984) 6851-6855; U.S. Pat. Nos. 5,202,238; 5,204,244). In some instances, framework region (FR) residues of the acceptor immunoglobulin are replaced by corresponding non-human residues. Furthermore, humanized immunoglobulins may comprise further modifications, e.g. amino acid residues that are not found in the acceptor immunoglobulin or in the donor immunoglobulin. Such modifications result in variants of such recipient or donor immunoglobulin, which are homologous but not identical to the corresponding parent sequence.

Methods for humanizing non-human immunoglobulin have been described in the art. Generally, a humanized immunoglobulin comprises one or more amino acid residues introduced into it from a source which is non-human. These non-human amino acid residues are often referred to as “import” residues, which are typically taken from an “import” variable domain. Humanization can be essentially performed following the method of Winter and co-workers by substituting hypervariable region sequences for the corresponding sequences of a non-human immunoglobulin (see e.g. Winter, G. and Harris, W. J., Immunol. Today 14 (1993) 243-246).

The term “human immunoglobulin” as used herein, denotes an immunoglobulin having variable and constant regions (domains) derived from human germ line immunoglobulin sequences and having high sequence similarity or identity with these germ line sequences. The variable heavy chain region is in one embodiment derived from germline sequence DP-50 (GenBank LO6618) and the variable light chain region is derived from germline sequence L6 (GenBank X01668) or the variable heavy chain region is derived DP-61 (GenBank M99682) and the variable light chain region is derived from germline sequence L15 (GenBank K01323). The constant regions of the antibody are constant regions of human IgG1 or IgG4 type or a variant thereof. Such regions can be allotypic and are described by, e.g., Johnson, G. and Wu, T. T., Nucleic Acids Res. 28 (2000) 214-218, and the databases referenced therein.

The term “recombinant immunoglobulin” as used herein denotes an immunoglobulin that is prepared, expressed, or created by recombinant means, such as immunoglobulins isolated from host cells, such as E.coli, NS0, BHK, or CHO cells, or from an animal (e.g. a mouse or rabbit) that is transgenic for human immunoglobulin genes. “Recombinant human immunoglobulins” according to the invention have in one embodiment variable and constant regions in a rearranged form. The recombinant human immunoglobulins according to the invention have been subjected to in vivo somatic hypermutation. Thus, the amino acid sequences of the VH and VL regions of the recombinant human immunoglobulins are sequences that can be assigned to defined human germ line VH and VL sequences, but may not naturally exist within the human antibody germ line repertoire in vivo.

The term “monoclonal immunoglobulin” as used herein refers to an immunoglobulin obtained from a population of substantially homogeneous immunoglobulins, i.e. the individual immunoglobulins of the population are identical except for naturally occurring mutations that may be present in minor amounts. Monoclonal immunoglobulins are highly specific, being directed against a single antigenic site. Furthermore, in contrast to polyclonal immunoglobulin preparations, which include different immunoglobulins directed against different antigenic sites (determinants or epitopes), each monoclonal immunoglobulin is directed against a single antigenic site. In addition to their specificity, the monoclonal immunoglobulins are advantageous in that they may be synthesized uncontaminated by other immunoglobulins. The modifier “monoclonal” indicates the character of the immunoglobulin as being obtained from a substantially homogeneous population of immunoglobulins and is not to be construed as requiring production of the immunoglobulin by any particular method.

Immunoglobulins having “conservative sequence modifications”, which are amino acid sequence modifications which do not affect or alter the characteristics of the immunoglobulin, are denoted as “variant immunoglobulins”. Modifications can be introduced by standard techniques known in the art, such as site-directed mutagenesis and PCR-mediated mutagenesis. Conservative amino acid substitutions include ones in which the amino acid residue is replaced with an amino acid residue having a similar side chain. These families include amino acids with basic side chains (e.g. lysine, arginine, histidine), acidic side chains (e.g. aspartic acid, glutamic acid), uncharged polar side chains (e.g. glycine, asparagine, glutamine, serine, threonine, tyrosine, cysteine, tryptophan), non-polar side chains (e.g. alanine, valine, leucine, isoleucine, proline, phenylalanine, methionine), beta-branched side chains (e.g. threonine, valine, isoleucine), and aromatic side chains (e.g. tyrosine, phenylalanine, tryptophan, histidine). Thus, a predicted amino acid residue not essential for antigen binding in an immunoglobulin can be replaced with another amino acid residue from the same side chain family.

The term “variable domain” (variable domain of a light chain (V_{L}), variable domain of a heavy chain (V_{H})) as used herein denotes each of the individual domains of a pair of light and heavy chains of an immunoglobulin which are directly involved in the binding of the target antigen. The variable domains are generally the N-terminal domains of light and heavy chains. The variable domains of the light and heavy chain have the same general structure, i.e. they possess an “immunoglobulin framework”, and each domain comprises four “framework regions” (FR), whose sequences are widely conserved, connected by three “hypervariable regions” (or “complementarity determining regions”, CDRs). The terms “complementary determining region”

(CDR) or “hypervariable region” (HVR), which are used interchangeably within the current application, denote the amino acid residues of an antibody which are mainly involved in antigen-binding. “Framework” regions (FR) are those variable domain regions other than the hypervariable regions. Therefore, the light and heavy chain variable domains of an immunoglobulin comprise from N- to C-terminus the regions FR1, CDR1, FR2, CDR2, FR3, CDR3, and FR4. The framework regions adopt a β-sheet conformation and the CDRs form loops connecting the β-sheet structure. The CDRs in each chain are held in their three-dimensional structure by the framework regions and form together with the CDRs from the other chain the antigen binding site. The immunoglobulin heavy and light chain CDR3 region plays a particularly important role in the binding specificity/affinity of the immunoglobulin. CDR and FR regions are determined according to the standard definition of Kabat, E. A., et al., Sequences of Proteins of Immunological Interest, 5th ed., Public Health Service, National Institutes of Health, Bethesda, MD (1991).

The term “amino acid” as used within this application denotes the group of carboxy α-amino acids, which directly or in form of a precursor can be encoded by nucleic acid. The individual amino acids are encoded by nucleic acids consisting of three nucleotides, so called codons or base-triplets. Each amino acid is encoded by at least one codon. The encoding of the same amino acid by different codons is known as “degeneration of the genetic code”. The term “amino acid” as used within this application denotes the naturally occurring carboxy α-amino acids and comprises alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

A “nucleic acid” or a “nucleic acid sequence”, which terms are used interchangeably within this application, refers to a polymeric molecule consisting of the individual nucleotides (also called bases) ‘a’, ‘c’, ‘g’, and ‘t’ (or ‘u’ in RNA), i.e. to DNA, RNA, or modifications thereof. This polynucleotide molecule can be a naturally occurring polynucleotide molecule or a synthetic polynucleotide molecule or a combination of one or more naturally occurring polynucleotide molecules with one or more synthetic polynucleotide molecules. Also encompassed by this definition are naturally occurring polynucleotide molecules in which one or more nucleotides are changed (e.g. by mutagenesis), deleted, or added. A nucleic acid can either be isolated, or integrated in another nucleic acid, e.g. in an expression cassette, a plasmid, or the chromosome of a host cell. A nucleic acid is characterized by its nucleic acid sequence consisting of individual nucleotides.

To a person skilled in the art procedures and methods are well known to convert an amino acid sequence, e.g. of a polypeptide, into a corresponding nucleic acid sequence encoding this amino acid sequence. Therefore, a nucleic acid is characterized by its nucleic acid sequence consisting of individual nucleotides and likewise by the amino acid sequence of a polypeptide encoded thereby.

It has now been found that a nucleic acid encoding a monoclonal immunoglobulin can be obtained from a single cell with a method according to the invention comprising a polymerase chain reaction (PCR). Further it has been found that with a combination of the PCR method according to the invention and an in vitro translation method the nucleic acid encoding a monoclonal immunoglobulin can be obtained from a single cell and the encoded immunoglobulin can be provided in quantities sufficient for the characterization of the immunoglobulin's binding properties. In order to amplify the very low amount of mRNA obtained from a single cell, the individual PCR (polymerase chain reaction) has to be very sensitive and a combination of more than one PCR has to be performed.

Thus, it has been found that based on the amplification of nucleic acid encoding cognate IgG HC (immunoglobulin G heavy chain) and IgG LC (immunoglobulin G light chain) of an IgG isotype immunoglobulin from a single cell with subsequent in vitro translation of the obtained amplified nucleic acid Fab fragments or complete immunoglobulins can be provided. With this method a high sensitive method for obtaining information about an immunoglobulin produced by a single cell is provided. This is possible even from the minute amounts of mRNA of a single cell. The method according to the invention allows for the biochemical characterization of the binding characteristics of an immunoglobulin expressed by a single B-cell. Thus, with this method characterization of a higher diversity as opposed to the hybridoma technology is possible. Furthermore, as the cognate immunoglobulin chains are obtained from mature B-cells after antigen contact, selectively the nucleic acids encoding high specific and correctly assembled immunoglobulins are obtained.

The method according to the current invention for obtaining the nucleic acid encoding an immunoglobulin form a single cell comprises a multiplex semi-nested PCR for the amplification of cognate IgG HC and IgG LC (human IgG isotype) from a single B-cell. For characterization of the binding characteristics of the immunoglobulin encoded by the obtained nucleic acid, the PCR product was transformed to a nucleic acid encoding the corresponding Fab-fragment. Thereafter the Fab-fragment was translated in vitro in E. coli lysate. The expression was confirmed using ELISA and Western blot methods.

In general one aspect of the current invention is a method employing the following steps i) isolating with magnetic micro-beads coated with human CD19 B-cells from peripheral blood, ii) depositing single cells e.g. by limited dilution or FACS, iii) extracting the mRNA of the individualized B-cells, iv) obtaining one or more nucleic acids encoding at least the variable domains (VH and VL) of the immunoglobulin produced by the individualized B-cell, v) translating in vitro a linear RNA template, and optionally vi) characterizing the binding properties of the immunoglobulin or immunoglobulin fragment.

The IgG-specific PCR amplification according to the current invention was optimized and modified resulting in an increase in determined OD-values and, thus, obtained immunoglobulin or immunoglobulin fragment after in vitro translation.

Three novel PCR-based approaches were established which are highly sensitive and result in high recovery of the amplified nucleic acids encoding the immunoglobulin's heavy and light chains or fragments thereof. Also provided is a method for the expression of functional and stable Fab fragments after in vitro translation of nucleic acid obtained with the PCR-based method according to the invention.

The terms “polymerase chain reaction” and “PCR”, which are used interchangeably in this application, denote a method for specifically amplifying a region of nucleic acids, e.g. of DNA or RNA. This method has been developed by K. Mullis (see e.g. Winkler, M. E., et al., Proc. Natl. Acad. Sci. USA 79 (1982) 2181-2185). The region can be a single gene, a part of a gene, a coding or a non-coding sequence. Most PCR methods typically amplify DNA fragments of hundreds of base pairs (bp), although some techniques allow for amplification of fragments up to 40 kilo base pairs (kb) in size. A basic PCR set up requires several components and reagents. These components include a nucleic acid template that contains the region to be amplified, two primer complementary to the 5′-and 3′-end of the region to be amplified, a polymerase, such as Taq polymerase or another thermostable polymerase, deoxynucleotide triphosphates (dNTPs) from which the polymerase synthesizes a new strand, a buffer solution providing a suitable chemical environment for optimum activity and stability of the polymerase, divalent cations, generally Mg²⁺, and finally, monovalent cations like potassium ions.

The term “semi-nested PCR” denotes two successive polymerase chain reactions each employing at least a pair of PCR primer, wherein in the first polymerase chain reaction a first pair of primer is employed and in the second polymerase chain reaction a second pair of primer is employed. In the first and second pair of primer one of the primer is the same and the other primer is changed, whereby the distance, i.e. the number of nucleotides, between the 3′-end of the first primer and the 5′-end of the second primer is reduced in the pair of primer used in the second polymerase chain reaction compared to the pair of primer used in the first polymerase chain reaction. The changed primer is either the sense primer or the anti-sense primer. The first PCR amplifies a sequence as seen in any PCR experiment. One primer of the second pair of primer, i.e. the nested primer, for the second PCR binds within the first PCR product and produces a second PCR product that is shorter than the first one. The technique, because it uses four specific primer, rather than two, has greater specificity than regular PCR. It can also yield detectable product in cases where simple PCR fails to do so.

The terms “multiplex polymerase chain reaction” or “multiplex PCR”, which are used interchangeably within the current application, denote a polymerase chain reaction employing multiple, unique primer in a single PCR reaction/mixture to produce amplicons of varying sizes specific to different DNA sequences. By targeting multiple genes at once, additional information can be obtained from a single test run that otherwise would require several times the reagents and more time to perform. Annealing temperatures for each primer sets must be optimized to work correctly within a single reaction. Besides, amplicon sizes should be different enough to form distinct bands when visualized by gel electrophoresis.

In the human genome the chromosomal loci containing the immunoglobulin encoding genes are located on chromosomes 2, 14, and 22 (see FIGS. 1A-C). The human immunoglobulin G heavy chain locus can be found on chromosome 14 (14q32.2) with the chromosomal orientation in the locus: telomere-5′-end-V_{H}-D-J_{H}-C_{H}-3′-end—centromere. The V_{H} segments on the chromosome are classified as depicted in the following Table 1.

TABLE 1
Grouping of the V_{H}-genes into V_{H} families according to Matsuda,
F., et al., J. Exp. Med. 188 (1998) 2151-2162 and Tomlinson,
I. M., et al., V Base sequence directory 1999.

| V_{H} | Number of family | Genes with open reading |
|---|---|---|
| family | members | frame |
| V_{H}1 | 14 | 9/11 |
| V_{H}2 | 4 | 3 |
| V_{H}3 | 65 | 22 |
| V_{H}4 | 32 | 7/11 |
| V_{H}5 | 2 | 2 |
| V_{H}6 | 1 | 1 |
| V_{H}7 | 5 | 1 |

The human immunoglobulin G heavy chain locus comprises overall 123-129

V_{H}-genes, of which 51 are functional, 23 functional D-genes (D=diversity), grouped in seven families, 6 functional J_{H}-genes (J=joining) and in the most frequent haplotype 9 functional C_{H}-genes (C=constant).

The locus for the human immunoglobulin G light chains of the types kappa (κ) and lambda (λ) is located on two different chromosomes, chromosomes 2 and 22. The kappa light chain locus can be found on the short arm of chromosome 2 (2p11.2) and comprises 40 functional V_{κ}-gene segments. These are grouped in seven families. The locus also comprises 5 J_{κ}-genes and a single C_{κ}-gene (Schable, K. F. and Zachau, H. G., Biol. Chem. Hoppe Seyler 374 (1993) 1001-1022; Lefranc, M. P., Exp. Clin. Immunogenet. 18 (2001) 161-174).

TABLE 2
Grouping of the V_{κ}-genes into V_{κ} families according to Foster,
S. J., et al., J. Clin. Invest. 99 (1997) 1614-1627.

| V_{κ} | Number of |
|---|---|
| family | functional genes |
| V_{κ}1 | 19 |
| V_{κ}2 | 9 |
| V_{κ}3 | 7 |
| V_{κ}4 | 1 |
| V_{κ}5 | 1 |
| V_{κ}6 | 3 |

The lambda light chain locus can be found on the long arm of chromosome 22 (22p11.2) and comprises 73-74 V_{λ}-gene of which 30 are functional. These are grouped in ten families which in addition are grouped in three clusters. The locus also comprises 7 J_{λ}-genes, of which 5 are functional.

TABLE 3
Grouping of the V_{λ}-genes into V_{λ} families according
to Frippiat, J. P., et al., Hum. Mol. Genet. 4 (1995) 983-991;
Farner, N. L., et al., J. Immunol. 162 (1999) 2137-2145; Lefranc,
M. P., Exp. Clin. Immunogenet. 18 (2001) 242-254.

| V_{λ} | Number of | |
|---|---|---|
| family | functional genes | Cluster |
| V_{λ}1 | 5 | B |
| V_{λ}2 | 5 | A |
| V_{λ}3 | 8 | A |
| V_{λ}4 | 3 | A-C |
| V_{λ}5 | 3 | B |
| V_{λ}6 | 1 | C |
| V_{λ}7 | 2 | B |
| V_{λ}8 | 1 | C |
| V_{λ}9 | 1 | B |
| V_{λ}10 | 1 | C |

The PCR-based amplification of the nucleic acid encoding an IgG HC and LC or at least the variable domain thereof from a single immunoglobulin producing cell, e.g. from a single B-cell, is based on the single cell deposition of B-lymphocytes followed by a PCR based nucleic acid amplification with specific primer for the variable domain of the heavy and light chain. The outcome of the PCR is essentially depending on the employed PCR primer. At best the employed primer should cover all V-genes, should not be prone to dimer formation and should specifically bind to the cDNA encoding the immunoglobulin. Thus, in one embodiment the nucleic acid encoding an immunoglobulin variable domain is obtained from cDNA.

Due to the large number of functional genes on the human immunoglobulin G locus it is necessary to employ different primer in the PCR reaction in order to cover as many known genes as possible. Therefore, a set of degenerated primer has been established which is also an aspect of the current invention. In one embodiment the amplification of the nucleic acid encoding the heavy and light chain is performed in one polymerase chain reaction. In this embodiment the primer are chosen in order to provide for the amplification of nucleic acids of approximately the same length in order to allow for the same PCR conditions. In this embodiment primer for the nucleic acid encoding the heavy chain are employed whereof one is binding in the heavy chain C_{H}1 region, thus, providing for a nucleic acid fragment of comparable size to that of the corresponding nucleic acid encoding the light chain.

One aspect of the current invention is a method for obtaining a nucleic acid encoding at least an immunoglobulin variable domain from a single cell comprising the following steps:
obtaining a first nucleic acid composition by performing a first polymerase chain reaction with three to six 5′-primer and one 3′-primer,
obtaining a nucleic acid encoding an immunoglobulin variable domain by performing with the composition obtained in the first polymerase chain reaction a second polymerase chain reaction with thirteen to sixteen 5′-primer and one 3′-primer,
whereby the distance of the binding locations of the primer employed in the second polymerase chain reaction is reduced compared to the first polymerase chain reaction.

In one embodiment of this method the 5′-primer employed in the first polymerase chain reaction bind in the coding region for the leader peptide of the immunoglobulin. In another embodiment the 5′-primer employed in the second polymerase chain reaction bind in the coding region for the first framework region of the immunoglobulin. In another embodiment the primer employed in the second polymerase chain reaction provide for overhangs encoding the translational start codon ATG for 5′-primer and/or the translational stop codon TTA for 3′-primer. This overhang is useful in an optional following overlapping polymerase chain reaction for the generation of nucleic acids for the in vitro translation of the obtained nucleic acid. In one embodiment the immunoglobulin variable domain is an immunoglobulin heavy chain variable domain or an immunoglobulin kappa light chain variable domain or an immunoglobulin lambda light chain variable domain.

In one embodiment of the methods according to the invention the primer employed in the first polymerase chain reaction for obtaining a nucleic acid encoding an immunoglobulin heavy chain variable domain have the nucleic acid sequence of SEQ ID NO: 05 and/or 06, and SEQ ID NO: 07 and/or 08, and SEQ ID NO: 09, and SEQ ID NO: 10 and/or 11, and SEQ ID NO: 12, and SEQ ID NO: 13, and SEQ ID NO: 14 and/or 15.

TABLE 4
Primer employed in the first polymerase chain reaction for
obtaining a nucleic acid encoding an immunoglobulin heavy chain
variable domain.

| Primer | | | SEQ ID |
|---|---|---|---|
| description | Sequence | Denotation | NO: |
| V_{H} Primer | TCACCATGGACTG(C/G)ACCTGGA | V_{H}L-1 | 05, 06 |
| binding in | CCATGGACACACTTTG(C/T)TCCAC | V_{H}L-2 | 07, 08 |
| the leader | TCACCATGGAGTTTGGGCTGAGC | V_{H}L-3 | 09 |
| peptide | AGAACATGAAACA(C/T)CTGTGGTTC | V_{H}L-4 | 10, 11 |
| coding | TT | | |
| region | ATGGGGTCAACCGCCATCCT | V_{H}L-5 | 12 |
| | ACAATGTCTGTCTCCTTCCTCAT | V_{H}L-6 | 13 |
| primer | GCCAGGGGGAAGAC(C/G)GATG | huC_{H}-II | 14, 15 |
| binding in | | | |
| the | | | |
| constant | | | |
| region | | | |
| coding | | | |
| region | | | |

In one embodiment of the methods according to the invention the primer employed in the first polymerase chain reaction for obtaining a nucleic acid encoding an immunoglobulin kappa light chain variable domain have the nucleic acid sequence of SEQ ID NO: 16 to 20.

TABLE 5
Primer employed in the first polymerase chain reaction for
obtaining a nucleic acid encoding an immunoglobulin kappa light
chain variable domain.

| Primer | | | SEQ |
|---|---|---|---|
| description | Sequence | Denotation | ID NO: |
| V_{κ} primer | GCTCAGCTCCTGGGGCTCCTG | V_{κ}L-1 | 16 |
| binding in the | CTGGGGCTGCTAATGCTCTGG | V_{κ}L-2 | 17 |
| leader peptide | TTCCTCCTGCTACTCTGGCTC | V_{κ}L-3 | 18 |
| coding region | CAGACCCAGGTCTTCATTTCT | V_{κ}L-4 | 19 |
| primer binding in | TTTCAACTGCTCATCAGATGGCGG | huC_{κ}-II | 20 |
| the constant | | | |
| region coding | | | |
| region | | | |

In one embodiment of the methods according to the invention the primer employed in the first polymerase chain reaction for obtaining a nucleic acid encoding an immunoglobulin lambda light chain variable domain have the nucleic acid sequence of SEQ ID NO: 21, and SEQ ID NO: 22, and SEQ ID NO: 23 and/or 24 and/or 25 and/or 26, and SEQ ID NO: 27 and/or 28.

TABLE 6
Primer employed in the first polymerase chain reaction for
obtaining a nucleic acid encoding an immunoglobulin lambda
light chain variable domain.

| Primer | | | SEQ |
|---|---|---|---|
| description | sequence | denotation | ID NO: |
| V_{λ} primer | CCTCTCCTCCTCACCCTCCT | V_{λ}L-1 | 21 |
| binding in the | CTCCTCACTCAGGGCACA | V_{λ}L-2 | 22 |
| leader peptide | ATGGCCTGGA(T/C)C(C/G)CTCTCC | V_{λ}L-3 | 23, 24, |
| coding region | | | 25, 26 |
| primer binding in | AGCTCCTCAGAGGAGGG(C/T)GG | C_{λ}II | 27, 28 |
| the constant | | | |
| region coding | | | |
| region | | | |

In one embodiment of the methods according to the invention the primer employed in the second polymerase chain reaction for obtaining a nucleic acid encoding an immunoglobulin heavy chain variable domain have the nucleic acid sequence of SEQ ID NO: 29 and/or 30, and SEQ ID NO: 31, and SEQ ID NO: 32 and/or 33, and SEQ ID NO: 34 and/or 35, and SEQ ID NO: 36, and SEQ ID NO: 37 and/or 38, and SEQ ID NO: 39 and/or 40, and SEQ ID NO: 41, and SEQ ID NO: 42, and SEQ ID NO: 43 and/or 44, and SEQ ID NO: 45, and SEQ ID NO: 46 and/or 47, and SEQ ID NO: 48, and SEQ ID NO: 49 and/or 50, and SEQ ID NO: 51 and/or 52.

TABLE 7
Primer employed in the second polymerase chain reaction for
obtaining a nucleic acid encoding an immunoglobulin heavy chain
variable domain.

| | | | SEQ |
|---|---|---|---|
| Primer | | | ID |
| description | Sequence | Denotation | NO: |
| V_{H} primer | CTTTAAGAAGGAGATATACCATGGT | V_{H}L-1a | 29, 30 |
| binding in the | (G/T)CAGCTGGTGCAG | | |
| FR1 coding | CTTTAAGAAGGAGATATACCATGCA | V_{H}L-1b | 31 |
| region | GGTCCAGCTTGTGCAG | | |
| | CTTTAAGAAGGAGATATACCATG | V_{H}L-1c | 32, 33 |
| | (G/C)AGGTCCAGCTGGTACAG | | |
| | CTTTAAGAAGGAGATATACCATGCA | V_{H}L-1d | 34, 35 |
| | (A/G)ATGCAGCTGGTGCAG | | |
| | CTTTAAGAAGGAGATATACCATGCA | V_{H}L-2a | 36 |
| | G ATCACCTTGAAGGAG | | |
| | CTTTAAGAAGGAGATATACCATGCA | V_{H}L-2b | 37, 38 |
| | G GTCACCTTGA(A/G)GGAG | | |
| | CTTTAAGAAGGAGATATACCATGGA | V_{H}L-3a | 39, 40 |
| | (A/G)GTGCAGCTGGTGGAG | | |
| | CTTTAAGAAGGAGATATACCATGCA | V_{H}L-3b | 41 |
| | G GTGCAGCTGGTGGAG | | |
| | CTTTAAGAAGGAGATATACCATGGA | V_{H}L-3c | 42 |
| | G GTGCAGCTGTTGGAG | | |
| | CTTTAAGAAGGAGATATACCATGCA | V_{H}L-4a | 43,44 |
| | G(C/G)TGCAGCTGCAGGAG | | |
| | CTTTAAGAAGGAGATATACCATGCA | V_{H}L-4b | 45 |
| | G GTGCAGCTACAGCAG | | |
| | CTTTAAGAAGGAGATATACCATGGA | V_{H}L-5a | 46, 47 |
| | (A/G)GTGCAGCTGGTGCAG | | |
| | CTTTAAGAAGGAGATATACCATGCA | V_{H}L-6a | 48 |
| | GGTACAGCTGCAGCAG | | |
| | CTTTAAGAAGGAGATATACCATGCA | V_{H}L-7a | 49, 50 |
| | GGT(C/G)CAGCTGGTGCAA | | |
| primer | ATCGTATGGGTAGCTGGTCCCTTAG | huC_{H}-III | 51, 52 |
| binding in the | AC(C/G)GATGGGCCCTTGGTGGA | | |
| constant | | | |
| region coding | | | |
| region | | | |

In one embodiment of the methods according to the invention the primer employed in the second polymerase chain reaction for obtaining a nucleic acid encoding an immunoglobulin kappa light chain variable domain have the nucleic acid sequence of SEQ ID NO: 53 and/or 54, and SEQ ID NO: 55 and/or 56, and SEQ ID NO: 57 and/or 58, and SEQ ID NO: 59, and SEQ ID NO: 60, and SEQ ID NO: 61 and/or 62, and SEQ ID NO: 63 and/or 64, and SEQ ID NO: 65, and SEQ ID NO: 66, and SEQ ID NO: 67, and SEQ ID NO: 68, and SEQ ID NO: 69, and SEQ ID NO: 70, and SEQ ID NO: 71.

TABLE 8
Primer employed in the second polymerase chain reaction for
obtaining a nucleic acid encoding an immunoglobulin kappa light
chain variable domain.

| Primer | | | SEQ |
|---|---|---|---|
| description | Sequence | Denotation | ID NO: |
| V_{κ} primer | CTTTAAGAAGGAGATATACCATG | V_{κ}L-1a | 53, 54 |
| binding in the | (A/G)ACATCCAGATGACCCAG | | |
| FR1 coding | CTTTAAGAAGGAGATATACCATGG | V_{κ}L-1b | 55, 56 |
| region | (A/C)CATCCAGTTGACCCAG | | |
| | CTTTAAGAAGGAGATATACCATGGC | V_{κ}L-1c | 57, 58 |
| | CATCC(A/G)GATGACCCAG | | |
| | CTTTAAGAAGGAGATATACCATGGT | V_{κ}L-1d | 59 |
| | CATCTGGATGACCCAG | | |
| | CTTTAAGAAGGAGATATACCATGGA | V_{κ}L-2a | 60 |
| | TATTGTGATGACCCAG | | |
| | CTTTAAGAAGGAGATATACCATGG | V_{κ}L-2b | 61, 62 |
| | AT(A/G)TTGTGATGACTCAG | | |
| | CTTTAAGAAGGAGATATACCATGG | V_{κ}L-3a | 63, 64 |
| | AAATTGTGTTGAC(A/G)CAG | | |
| | CTTTAAGAAGGAGATATACCATGG | V_{κ}L-3b | 65 |
| | AAATAGTGATGACGCAG | | |
| | CTTTAAGAAGGAGATATACCATGG | V_{κ}L-3c | 66 |
| | AAATTGTAATGACACAG | | |
| | CTTTAAGAAGGAGATATACCATGG | V_{κ}L-4a | 67 |
| | ACATCGTGATGACCCAG | | |
| | CTTTAAGAAGGAGATATACCATGG | V_{κ}L-5a | 68 |
| | AAACGACACTCACGCAG | | |
| | CTTTAAGAAGGAGATATACCATGG | V_{κ}L-6a | 69 |
| | AAATTGTGCTCACTCAG | | |
| | CTTTAAGAAGGAGATATACCATGGA | V_{κ}L-6b | 70 |
| | TGTTGTGATGACACAG | | |
| primer binding | ATCGTATGGGTAGCTGGTCCCTTAA | huC_{κ}-III | 71 |
| in the constant | AGATGAAGACAGATGGTGC | | |
| region coding | | | |
| region | | | |

In one embodiment of the methods according to the invention the primer employed in the second polymerase chain reaction for obtaining a nucleic acid encoding an immunoglobulin lambda light chain variable domain have the nucleic acid sequence of SEQ ID NO: 72, and SEQ ID NO: 73 and/or 74, and SEQ ID NO: 75, and SEQ ID NO: 76, and SEQ ID NO: 77 and/or 78, and SEQ ID NO: 79, and SEQ ID NO: 80, and SEQ ID NO: 81, and SEQ ID NO: 82 and/or 83, and SEQ ID NO: 84 and/or 85, and SEQ ID NO: 86, and SEQ ID NO: 87 and/or 88, and SEQ ID NO: 89, and SEQ ID NO: 90 and/or 91, and SEQ ID NO: 92, and SEQ ID NO: 93.

TABLE 9
Primer employed in the second polymerase chain reaction for
obtaining a nucleic acid encoding an immunoglobulin lambda
light chain variable domain.

| Primer | | | SEQ |
|---|---|---|---|
| description | Sequence | Denotation | ID NO: |
| V_{λ} primer | CTTTAAGAAGGAGATATACCATGCA | V_{λ}L-1a | 72 |
| binding in the | GTCTGTGCTGACTCAG | | |
| FR1 coding | CTTTAAGAAGGAGATATACCATGCA | V_{λ}L-1b | 73, 74 |
| region | GTCTGTG(C/T)TGACGCAG | | |
| | CTTTAAGAAGGAGATATACCATGCA | V_{λ}L-1c | 75 |
| | GTCTGTCGTGACGCAG | | |
| | CTTTAAGAAGGAGATATACCATGCA | V_{λ}L-2a | 76 |
| | GTCTGCCCTGACTCAG | | |
| | CTTTAAGAAGGAGATATACCATGTC | V_{λ}L-3a | 77, 78 |
| | CTATG(A/T)GCTGACTCAG | | |
| | CTTTAAGAAGGAGATATACCATGTC | V_{λ}L-3b | 79 |
| | CTATGAGCTGACACAG | | |
| | CTTTAAGAAGGAGATATACCATGTC | V_{λ}L-3c | 80 |
| | TTCTGAGCTGACTCAG | | |
| | CTTTAAGAAGGAGATATACCATGTC | V_{λ}L-3d | 81 |
| | CTATGAGCTGATGCAG | | |
| | CTTTAAGAAGGAGATATACCATGCA | V_{λ}L-4a | 82, 83 |
| | GC(C/T)TGTGCTGACTCAA | | |
| | CTTTAAGAAGGAGATATACCATGCAG | V_{λ}L-5a | 84, 85 |
| | (C/G)CTGTGCTGACTCAG | | |
| | CTTTAAGAAGGAGATATACCATGAA | V_{λ}L-6a | 86 |
| | TTTTATGCTGACTCAG | | |
| | CTTTAAGAAGGAGATATACCATGCAG | V_{λ}L-7a | 87, 88 |
| | (A/G)CTGTGGTGACTCAG | | |
| | CTTTAAGAAGGAGATATACCATGCAG | V_{λ}L-8a | 89 |
| | ACTGTGGTGACCCAG | | |
| | CTTTAAGAAGGAGATATACCATGC | V_{λ}L-4/9a | 90, 91 |
| | (A/T)GCCTGTGCTGACTCAG | | |
| | CTTTAAGAAGGAGATATACCATGCAG | V_{λ}L-10a | 92 |
| | GCAGGGCTGACTCAG | | |
| primer binding | ATCGTATGGGTAGCTGGTCCCTTAG | huC_{λ}-III | 93 |
| in the constant | GGAACAGAGTGACCG | | |
| region coding | | | |
| region | | | |

A further aspect of the current invention is a method for obtaining a nucleic acid encoding at least an immunoglobulin variable domain from a single cell comprising the following step:
obtaining a first nucleic acid composition by performing a first polymerase chain reaction with three or four 5′-primer and one 3′-primer,
obtaining a nucleic acid encoding at least an immunoglobulin variable domain by performing with the composition obtained in the first polymerase chain reaction a second polymerase chain reaction with one 5′-primer and one 3′-primer,
whereby primer employed in the first and in the second polymerase chain reaction can be the same.

In one embodiment the primer employed in the polymerase chain reaction provide for overhangs encoding the translational start codon ATG for 5′-primer and/or the translational stop codon TTA for 3′-primer. This overhang is useful in an optional following overlapping polymerase chain reaction for the generation of nucleic acids for the in vitro translation of the obtained nucleic acid. In one embodiment the immunoglobulin variable domain is an immunoglobulin heavy chain variable domain or an immunoglobulin kappa light chain variable domain or an immunoglobulin lambda light chain variable domain.

In one embodiment of this method the primer employed in the first two-step polymerase chain reaction for obtaining a nucleic acid encoding an immunoglobulin heavy chain variable domain have the nucleic acid sequence of SEQ ID NO: 94, and SEQ ID NO: 95, and SEQ ID NO: 96 and/or 97 and/or 98 and/or 99, and SEQ ID NO: 100 and/or 101 and/or 102 and/or 103, and SEQ ID NO: 104 and/or 105 and/or 106.

TABLE 10
Primer employed in the first two-step polymerase chain reaction for
obtaining a nucleic acid encoding an immunoglobulin heavy chain
variable domain.

| Primer | | | SEQ ID |
|---|---|---|---|
| description | Sequence | Denotation | NO: |
| V_{H} primer | CTTTAAGAAGGAGATATACCATGCA | huV_{H}-1 | 94 |
| binding in | GGTGCAGCTGGTGCAGTC | | |
| the FR1 | CTTTAAGAAGGAGATATACCATGCA | huV_{H}-2 | 95 |
| coding | GGTCAACTTAAGGGAGTCTGG | | |
| region | CTTTAAGAAGGAGATATACCATGAG | huV_{H}-3 | 96, 97, |
| | GTGCAGCTG(C/G)TG(C/G)AGTC | | 98, 99 |
| | CTTTAAGAAGGAGATATACCATGCA | huV_{H}-4 | 100, 101, |
| | GGT(A/G)CAGCTGCAG(C/G)AGTC | | 102, 103 |
| primer | ATCGTATGGGTAGCTGGTCCCTTA | huC_{H}-2 | 104, 105, |
| binding in | GTGGTGGTGGTGGTGGTGAACT | | 106 |
| the constant | (C/G/T)TCTTGTCCACCTTGGTGTTG | | |
| region | | | |
| coding | | | |
| region | | | |

In one embodiment of this method the primer employed in the first two-step polymerase chain reaction for obtaining a nucleic acid encoding an immunoglobulin kappa light chain variable domain have the nucleic acid sequence of SEQ ID NO: 107 and/or 108 and/or 109 and/or 110, and SEQ ID NO: 111 and/or 112, and SEQ ID NO: 113, and SEQ ID NO: 114, and SEQ ID NO: 115.

TABLE 11
Primer employed in the first two-step polymerase chain reaction
for obtaining a nucleic acid encoding an immunoglobulin kappa
light chain variable domain.

| Primer | | | SEQ ID |
|---|---|---|---|
| description | Sequence | Denotation | NO: |
| V_{κ} primer | CTTTAAGAAGGAGATATACCATGG | huV_{κ}-1 | 107, 108, |
| binding in the | A CATC(C/G)(A/T)GATGACCCAGTCT | | 109, 110 |
| FR1 coding | CTTTAAGAAGGAGATATACCATGG | huV_{κ}-2 | 111, 112 |
| region | A TATTGTG(A/C)TGACTCAGTCTCC | | |
| | CTTTAAGAAGGAGATATACCATGG | huV_{κ}-3 | 113 |
| | A AATTGTGTTGACGCAGTCTCC | | |
| | CTTTAAGAAGGAGATATACCATGG | huV_{κ}-4 | 114 |
| | A AACGACACTCACGCAGTCTC | | |
| primer | ATCGTATGGGTAGCTGGTCCCTTAA | huC_{κ}-2 | 115 |
| binding in the | CACCTCTCCCCTGTTGAAGCTC | | |
| constant | | | |
| region coding | | | |
| region | | | |

In one embodiment of this method the primer employed in the first two-step polymerase chain reaction for obtaining a nucleic acid encoding an immunoglobulin lambda light chain variable domain have the nucleic acid sequence of SEQ ID NO: 116, and SEQ ID NO: 117, and SEQ ID NO: 118 and/or 119, and SEQ ID NO: 120 and/or 121 and/or 122 and/or 123 and/or 124 and/or 125.

TABLE 12
Primer employed in the first two-step polymerase chain reaction
for obtaining a nucleic acid encoding an immunoglobulin lambda
light chain variable domain.

| Primer | | | SEQ ID |
|---|---|---|---|
| description | Sequence | Denotation | NO: |
| V_{λ} primer | CTTTAAGAAGGAGATATACCATG | huV_{λ}-1 | 116 |
| binding in the | CAGTCTGTGCTGACTCAGCC | | |
| FR1 coding | CTTTAAGAAGGAGATATACCATG | huV_{λ}-2 | 117 |
| region | CAGTCTGCCCTGACTCAGCC | | |
| | CTTTAAGAAGGAGATATACCATG | huV_{λ}-3 | 118, 119 |
| | TCCTATGAGCTGAC(A/T)CAGCC | | |
| primer | ATCGTATGGGTAGCTGGTCCCTTA | huC_{λ}-2 | 120, 121, |
| binding in the | TGAACATTC(C/T)G(C/T)AGGGGC | | 122, 123, |
| constant | (A/T)ACT | | 124, 125 |
| region coding | | | |
| region | | | |

In one embodiment of this method the primer employed in the second two-step polymerase chain reaction have the nucleic acid sequence of SEQ ID NO: 126 and SEQ ID NO: 127.

TABLE 13
Primer employed in the second two-step polymerase chain reaction.

| Primer | | | SEQ ID |
|---|---|---|---|
| description | Sequence | Denotation | NO: |
| first primer | CTTTAAGAAGGAGATATACCATG | LTGS-lfp | 126 |
| second primer | ATCGTATGGGTAGCTGG | LTGS-rfp | 127 |

Another aspect of the current invention is a method for obtaining a nucleic acid encoding at least an immunoglobulin variable domain from a single cell comprising the following steps:
obtaining a first nucleic acid composition by performing a first polymerase chain reaction with four to six 5′-primer and one 3′-primer,
obtaining a nucleic acid encoding at least an immunoglobulin variable domain by performing with the composition obtained in the first polymerase chain reaction a second polymerase chain reaction with thirteen to fifteen 5′-primer and one 3′-primer,
whereby in the second polymerase chain reaction either the 5′-primer are the same as in the first polymerase chain reaction and the 3′-primer is different or the 3′-primer is the same as in the first polymerase chain reaction and at least one 5′-primer is different, and
whereby in the second polymerase chain reaction the number of nucleotides between the 5′-end of each of the 5′-primer and the 3′-end of the 3′-primer is smaller compared to the number of nucleotides between the 5′-end of each of the 5′-primer and the 3′-end of the 3′-primer in the first polymerase chain reaction.

In one embodiment of this method the 5′-primer employed in the first polymerase chain reaction bind in the coding region for the leader peptide of the immunoglobulin. In another embodiment the 5′-primer employed in the second polymerase chain reaction bind in the coding region for the first framework region of the immunoglobulin. In another embodiment the primer employed in the second polymerase chain reaction provide for overhangs encoding the translational start codon ATG for 5′-primer and/or the translational stop codon TTA for 3′-primer. This overhang is useful in an optional following overlapping polymerase chain reaction for the generation of nucleic acids for the in vitro translation of the obtained nucleic acid. In one embodiment of this method for obtaining an immunoglobulin variable domain encoding nucleic acid six 5′-primer and one 3′-primer are employed in the first polymerase chain reaction. In another embodiment four 5′-primer and one 3′-primer are employed in the second polymerase chain reaction. In one embodiment the immunoglobulin variable domain is an immunoglobulin heavy chain variable domain or an immunoglobulin kappa light chain variable domain or an immunoglobulin lambda light chain variable domain.

In one embodiment of the methods according to the invention the primer employed in the first polymerase chain reaction for obtaining a nucleic acid encoding an immunoglobulin heavy chain variable domain have the nucleic acid sequence of SEQ ID NO: 05 and/or 06, and SEQ ID NO: 07 and/or 08, and SEQ ID NO: 09, and SEQ ID NO: 10 and/or 11, and SEQ ID NO: 12, and SEQ ID NO: 13, and SEQ ID NO: 104 and/or 105 and/or 106.

TABLE 14
Primer employed in the first polymerase chain reaction for
obtaining a nucleic acid encoding an immunoglobulin heavy
chain variable domain.

| | | | SEQ |
|---|---|---|---|
| Primer | | | ID |
| description | Sequence | Denotation | NO: |
| V_{H} primer | TCACCATGGACTG(C/G)ACCTGGA | V_{H}L-1 | 05, 06 |
| binding in the | CCATGGACACACTTTG(C/T)TCCAC | V_{H}L-2 | 07, 08 |
| leader | TCACCATGGAGTTTGGGCTGAGC | V_{H}L-3 | 09 |
| peptide | AGAACATGAAACA(C/T)CTGTGGTTCTT | V_{H}L-4 | 10, 11 |
| coding region | ATGGGGTCAACCGCCATCCT | V_{H}L-5 | 12 |
| | ACAATGTCTGTCTCCTTCCTCAT | V_{H}L-6 | 13 |
| primer | TCGTATGGGTAGCTGGTCCCTTAGTGGT | huC_{H}-2 | 104, |
| binding in the | GGTGGTGGTGGTGAACT(C/G/T)TCTTGT | | 105, |
| constant | CCACCTTGGTGTTG | | 106 |
| region coding | | | |
| region | | | |

In one embodiment of the methods according to the invention the primer employed in the first polymerase chain reaction for obtaining a nucleic acid encoding an immunoglobulin kappa light chain variable domain have the nucleic acid sequence of SEQ ID NO: 16, and SEQ ID NO: 17, and SEQ ID NO: 18, and SEQ ID NO: 19, and SEQ ID NO: 115.

TABLE 15
Primer employed in the first polymerase chain reaction for
obtaining a nucleic acid encoding an immunoglobulin kappa light
chain variable domain.

| Primer | | | SEQ ID |
|---|---|---|---|
| description | Sequence | Denotation | NO: |
| V_{κ} primer | GCTCAGCTCCTGGGGCTCCTG | V_{κ}L-1 | 16 |
| binding in the | CTGGGGCTGCTAATGCTCTGG | V_{κ}L-2 | 17 |
| leader peptide | TTCCTCCTGCTACTCTGGCTC | V_{κ}L-3 | 18 |
| coding region | CAGACCCAGGTCTTCATTTCT | V_{κ}L-4 | 19 |
| primer binding in | ATCGTATGGGTAGCTGGTCCC | huC_{κ}-2 | 115 |
| the constant | TTAACACTCTCCCCTGTTGAA | | |
| region coding | GCTC | | |
| region | | | |

In one embodiment of the methods according to the invention the primer employed in the first polymerase chain reaction for obtaining a nucleic acid encoding an immunoglobulin lambda light chain variable domain have the nucleic acid sequence of SEQ ID NO: 21, and SEQ ID NO: 22, and SEQ ID NO: 23 and/or 24 and/or 25 and/or 26, and SEQ ID NO: 120 and/or 121 and/or 122 and/or 123 and/or 124 and/or 125.

TABLE 16
Primer employed in the first polymerase chain reaction for
obtaining a nucleic acid encoding an immunoglobulin lambda
light chain variable domain.

| Primer | | | SEQ ID |
|---|---|---|---|
| description | Sequence | Denotation | NO: |
| V_{λ} primer | CCTCTCCTCCTCACCCTCCT | V_{λ}L-1 | 21 |
| binding in the | CTCCTCACTCAGGGCACA | V_{λ}L-2 | 22 |
| leader peptide | ATGGCCTGGA(T/C)C(C/G)CTCTCC | V_{λ}L-3 | 23, 24, 25, |
| coding region | | | 26 |
| primer binding | ATCGTATGGGTAGCTGGTCCCTTA | huC_{λ}-2 | 120, 121, |
| in the constant | TGAACATTC(C/T)G(C/T)AGGGGC | | 122, 123, |
| region coding | (A/T)ACT | | 124, 125 |
| region | | | |

In one embodiment of the methods according to the invention the primer employed in the second polymerase chain reaction for obtaining a nucleic acid encoding an immunoglobulin heavy chain variable domain have the nucleic acid sequence of SEQ ID NO: 128, and SEQ ID NO: 129, and SEQ ID NO: 130, and SEQ ID NO: 131, and SEQ ID NO: 132, and SEQ ID NO: 133, and SEQ ID NO: 134, and SEQ ID NO: 135, and SEQ ID NO: 136, and SEQ ID NO: 137, and SEQ ID NO: 138, and SEQ ID NO: 139, and SEQ ID NO: 140, SEQ and ID NO: 141, and SEQ ID NO: 104 and/or 105 and/or 106.

TABLE 17
Primer employed in the second polymerase chain reaction for
obtaining a nucleic acid encoding an immunoglobulin heavy
chain variable domain.

| | | | SEQ |
|---|---|---|---|
| Primer | | | ID |
| description | Sequence | Denotation | NO: |
| V_{H} primer | CTTTAAGAAGGAGATATACCATGCA | V_{H}-1a | 128 |
| binding in the | GGTKCAGCTGGTGCAG | | |
| FR1 coding | CTTTAAGAAGGAGATATACCATGCA | V_{H}-1b | 129 |
| region | GGTCCAGCTTGTGCAG | | |
| | CTTTAAGAAGGAGATATACCATGSAG | V_{H}-1c | 130 |
| | GTCCAGCTGGTACAG | | |
| | CTTTAAGAAGGAGATATACCATGCA | V_{H}-1d | 131 |
| | RATGCAGCTGGTGCAG | | |
| | CTTTAAGAAGGAGATATACCATGCA | V_{H}-2a | 132 |
| | GATCACCTTGAAGGAG | | |
| | CTTTAAGAAGGAGATATACCATGCA | V_{H}-2b | 133 |
| | GGTCACCTTGARGGAG | | |
| | CTTTAAGAAGGAGATATACCATGGA | V_{H}-3a | 134 |
| | RGTGCAGCTGGTGGAG | | |
| | CTTTAAGAAGGAGATATACCATGCA | V_{H}-3b | 135 |
| | GGTGCAGCTGGTGGAG | | |
| | CTTTAAGAAGGAGATATACCATGGA | V_{H}-3c | 136 |
| | GGTGCAGCTGTTGGAG | | |
| | CTTTAAGAAGGAGATATACCATGCA | V_{H}-4a | 137 |
| | GSTGCAGCTGCAGGAG | | |
| | CTTTAAGAAGGAGATATACCATGCA | V_{H}-4b | 138 |
| | GGTGCAGCTACAGCAG | | |
| | CTTTAAGAAGGAGATATACCATGGA | V_{H}-5a | 139 |
| | RGTGCAGCTGGTGCAG | | |
| | CTTTAAGAAGGAGATATACCATGCA | V_{H}-6a | 140 |
| | GGTACAGCTGCAGCAG | | |
| | CTTTAAGAAGGAGATATACCATGCA | V_{H}-7a | 141 |
| | GGTACAGCTGGTGCAA | | |
| primer | ATCGTATGGGTAGCTGGTCCCTTAGT | huC_{H}-2 | 104, |
| binding in the | GGTGGTGGTGGTGGTGAACT(C/G/T)T | | 105, |
| constant | CTTGTCCACCTTGGTGTTG | | 106 |
| region coding | | | |
| region | | | |

In one embodiment of the methods according to the invention the primer employed in the second polymerase chain reaction for obtaining a nucleic acid encoding an immunoglobulin kappa light chain variable domain have the nucleic acid sequence of SEQ ID NO: 53 and/or 54, and SEQ ID NO: 55 and/or 56, and SEQ ID NO: 57 and/or 58, and SEQ ID NO: 59, and SEQ ID NO: 60, and SEQ ID NO: 61 and/or 62, SEQ ID NO: 63 and/or 64, and SEQ ID NO: 65, and SEQ ID NO: 66, and SEQ ID NO: 67, and SEQ ID NO: 68, and SEQ ID NO: 69, and SEQ ID NO: 70, and SEQ ID NO: 115.

TABLE 18
Primer employed in the second polymerase chain reaction for
obtaining a nucleic acid encoding an immunoglobulin kappa light
chain variable domain.

| | | | SEQ |
|---|---|---|---|
| Primer | | | ID |
| description | Sequence | Denotation | NO: |
| V_{κ} primer | CTTTAAGAAGGAGATATACCATG(A/G) | V_{κ}L-1a | 53, 54 |
| binding in the | ACATCCAGATGACCCAG | | |
| FR1 coding | CTTTAAGAAGGAGATATACCATGG(A/ | V_{κ}L-1b | 55, 56 |
| region | C)CATCCAGTTGACCCAG | | |
| | CTTTAAGAAGGAGATATACCATGGCC | V_{κ}L-1c | 57, 58 |
| | ATCC(A/G)GATGACCCAG | | |
| | CTTTAAGAAGGAGATATACCATGGTC | V_{κ}L-1d | 59 |
| | ATCTGGATGACCCAG | | |
| | CTTTAAGAAGGAGATATACCATGGAT | V_{κ}L-2a | 60 |
| | ATTGTGATGACCCAG | | |
| | CTTTAAGAAGGAGATATACCATGGAT | V_{κ}L-2b | 61, 62 |
| | (A/G)TTGTGATGACTCAG | | |
| | CTTTAAGAAGGAGATATACCATGGA | V_{κ}L-3a | 63, 64 |
| | AATTGTGTTGAC(A/G)CAG | | |
| | CTTTAAGAAGGAGATATACCATGGA | V_{κ}L-3b | 65 |
| | AATAGTGATGACGCAG | | |
| | CTTTAAGAAGGAGATATACCATGGA | V_{κ}L-3c | 66 |
| | AATTGTAATGACACAG | | |
| | CTTTAAGAAGGAGATATACCATGGA | V_{κ}L-4a | 67 |
| | CATCGTGATGACCCAG | | |
| | CTTTAAGAAGGAGATATACCATGGA | V_{κ}L-5a | 68 |
| | AACGACACTCACGCAG | | |
| | CTTTAAGAAGGAGATATACCATGGA | V_{κ}L-6a | 69 |
| | AATTGTGCTGACTCAG | | |
| | CTTTAAGAAGGAGATATACCATGGAT | V_{κ}L-6b | 70 |
| | GTTGTGATGACACAG | | |
| primer | ATCGTATGGGTAGCTGGTCCCTTAAC | huC_{κ}-2 | 115 |
| binding in the | CATCTCCCCTGTTGAAGCTC | | |
| constant | | | |
| region coding | | | |
| region | | | |

In one embodiment of the methods according to the invention the primer employed in the second polymerase chain reaction for obtaining a nucleic acid encoding an immunoglobulin lambda light chain variable domain have the nucleic acid sequence of SEQ ID NO: 72, and SEQ ID NO: 73 and/or 74, and SEQ ID NO: 75, and SEQ ID NO: 76, and SEQ ID NO: 77 and/or 78, and SEQ ID NO: 79, and SEQ ID NO: 80, and SEQ ID NO: 81, and SEQ ID NO: 82 and/or 83, and SEQ ID NO: 84 and/or 85, and SEQ ID NO: 86, and SEQ ID NO: 87 and/or 88, and SEQ ID NO: 89, and SEQ ID NO: 90 and/or 91, and SEQ ID NO: 92, and SEQ ID NO: 120 or 121 or 122 or 123 or 124 or 125.

TABLE 19
Primer employed in the second polymerase chain reaction for
obtaining a nucleic acid encoding an immunoglobulin lambda
light chain variable domain.

| | | | SEQ |
|---|---|---|---|
| Primer | | | ID |
| description | Sequence | Denotation | NO: |
| V_{λ} primer | CTTTAAGAAGGAGATATACCATGC | V_{λ}L-1a | 72 |
| binding in the | AGTCTGTGCTGACTCAG | | |
| FR1 coding | CTTTAAGAAGGAGATATACCATGC | V_{λ}L-1b | 73, 74 |
| region | AGTCTGTG(C/T)TGACGCAG | | |
| | CTTTAAGAAGGAGATATACCATGC | V_{λ}L-1c | 75 |
| | AGTCTGTCGTGACGCAG | | |
| | CTTTAAGAAGGAGATATACCATGC | V_{λ}L-2 | 76 |
| | AGTCTGCCCTGACTCAG | | |
| | CTTTAAGAAGGAGATATACCATGTC | V_{λ}L-3a | 77, 78 |
| | CTATG(A/T)GCTGACTCAG | | |
| | CTTTAAGAAGGAGATATACCATGTC | V_{λ}L-3b | 79 |
| | CTATGAGCTGACACAG | | |
| | CTTTAAGAAGGAGATATACCATGTC | V_{λ}L-3c | 80 |
| | TTCTGAGCTGACTCAG | | |
| | CTTTAAGAAGGAGATATACCATGTC | V_{λ}L-3d | 81 |
| | CTATGAGCTGATGCAG | | |
| | CTTTAAGAAGGAGATATACCATGC | V_{λ}L-4 | 82, 83 |
| | AGC(C/T)TGTGCTGACTCAA | | |
| | CTTTAAGAAGGAGATATACCATGC | V_{λ}L-5 | 84, 85 |
| | AG(C/G)CTGTGCTGACTCAG | | |
| | CTTTAAGAAGGAGATATACCATGA | V_{λ}L-6 | 86 |
| | ATTTTATGCTGACTCAG | | |
| | CTTTAAGAAGGAGATATACCATGC | V2_{λ}L-7 | 87, 88 |
| | AG(A/G)CTGTGGTGACTCAG | | |
| | CTTTAAGAAGGAGATATACCATGC | V_{λ}L-8 | 89 |
| | AGACTGTGGTGACCCAG | | |
| | CTTTAAGAAGGAGATATACCATGC | V_{λ}L-4/9 | 90, 91 |
| | (A/T)GCCTGTGCTGACTCAG | | |
| | CTTTAAGAAGGAGATATACCATGC | V_{λ}L-10 | 92 |
| | AGGCAGGGCTGACTCAG | | |
| primer | ATCGTATGGGTAGCTGGTCCCTTAT | huC_{λ}-2 | 120, |
| binding in the | GAACATTC(C/T)G(C/T)AGGGGC | | 121, |
| constant | (A/T)ACT | | 122, |
| region coding | | | 123, |
| region | | | 124, |
| | | | 125 |

In one embodiment of the methods according to the invention the nucleic acid encoding the light chain variable domain and nucleic acid encoding the heavy chain variable domain are obtained in one polymerase chain reaction by a combination of the different 5′-and 3′-primer in a single multiplex polymerase chain reaction.

Another aspect of the current invention is a method for obtaining a nucleic acid encoding at least an immunoglobulin variable domain from a single cell comprising the following step:
performing a reverse transcription and polymerase chain reaction in one step with a set of primer comprising one 5′-primer and one 3′-primer.

In one embodiment of this method the 5′-primer employed in the multiplex one tube reverse transcription gene specific primer polymerase chain reaction (RT-GSP-PCR) binds in the coding region for the first framework region of the immunoglobulin. In another embodiment the primer employed in the RT-GSP-PCR reaction provide for overhangs encoding the translational start codon ATG for the 5′-primer and/or the translational stop codon TTA for the 3′-primer. This overhang is useful in an optional following overlapping polymerase chain reaction for the generation of nucleic acids for the in vitro translation of the obtained nucleic acid. In one embodiment this method is for obtaining an immunoglobulin heavy chain variable domain with a RT-GSP-PCR reaction. In one embodiment the immunoglobulin variable domain is an immunoglobulin heavy chain variable domain or an immunoglobulin kappa light chain variable domain or an immunoglobulin lambda light chain variable domain.

In one embodiment the primer employed in the multiplex one tube RT-GSP-PCR for obtaining a nucleic acid encoding an immunoglobulin heavy chain variable domain have the nucleic acid sequence of SEQ ID NO: 142 and 143.

TABLE 20
Primer employed in the multiplex one tube RT-GSP-PCR reaction
for obtaining a nucleic acid encoding an immunoglobulin heavy
chain variable domain.

| Primer | | | SEQ ID |
|---|---|---|---|
| description | Sequence | Denotation | NO: |
| V_{H} primer | CTTTAAGAAGGAGATATACCAT | V_{H}-lfp | 142 |
| binding in the | GAACTBTCTTGTCCACCTTGGT | | |
| FR1 coding | GTTG | | |
| region | | | |
| primer binding in | ATCGTATGGGTAGCTGGTCCCTT | V_{H}-rfp | 143 |
| the constant | AAACTBTCTTGTCCACCTTGGTG | | |
| region coding | TTG | | |
| region | | | |

In one embodiment of the methods according to the invention the primer employed in the multiplex one tube RT-GSP-PCR for obtaining a nucleic acid encoding an immunoglobulin kappa light chain variable domain have the nucleic acid sequence of SEQ ID NO: 144 and 145.

TABLE 21
Primer employed in the multiplex one tube RT-GSP-PCR
reaction for obtaining a nucleic acid encoding an immunoglobulin
kappa light chain variable domain.

| Primer | | | SEQ ID |
|---|---|---|---|
| description | Sequence | Denotation | NO: |
| V_{κ} primer | CTTTAAGAAGGAGATATACCAT | VL(k)-1fp | 144 |
| binding in the | GACACTCTCCCCTGTTGAAGCTC | | |
| FR1 coding | | | |
| region | | | |
| primer binding | ATCGTATGGGTAGCTGGTCCCTT | VL(k)-rfp | 145 |
| in the constant | AACACTCTCCCCTGTTGAAGCTC | | |
| region coding | | | |
| region | | | |

Further it has been found that with a combination of the PCR method according to the invention and a cell-free in vitro translation system the nucleic acids encoding the cognate immunoglobulin VH and VL domains can be obtained from a single cell whereby the encoded immunoglobulin variable domain is provided as Fab fragment in quantities sufficient for the characterization of the immunoglobulin's binding properties. In order to amplify the very low amount of mRNA obtained from a single cell, the individual PCR (polymerase chain reaction) has to be very sensitive and a combination of more than one PCR has to be performed.

A “cell-free in vitro translation system” according to the invention denotes a cell-free lysate of a prokaryotic or eukaryotic, preferably of a prokaryotic, cell containing ribosomes, tRNA, ATP, CGTP, nucleotides, and amino acids. In one embodiment the prokaryote is E. coli.

Cell-free in vitro translation is a method which has been known in the state of the art for a long time. Spirin et al. developed in 1988 a continuous-flow cell-free (CFCF) translation and coupled transcription/translation system in which a relatively high amount of protein synthesis occurs (Spirin, A. S., et al., Science 242 (1988) 1162-1164). For such cell-free in vitro translation, cell lysates containing ribosomes were used for translation or transcription/translation. Such cell-free extracts from E. coli were developed by, for example, Zubay (Zubay, G., et al., Ann. Rev. Genetics 7 (1973) 267-287) and were used by Pratt (Pratt, J. M., et al., Nucleic Acids Research 9 (1981) 4459-4474; and Pratt, J. M., et al., Transcription and Translation: A Practical Approach, Hames and Higgins (eds.), 179-209, IRL Press, 1984). Further developments of the cell-free protein synthesis are reported in U.S. Pat. Nos. 5,478,730, 5,571,690, EP 0 932 664, WO 99/50436, WO 00/58493, and WO 00/55353.

Eukaryotic cell-free expression systems are reported by, for example, Skup, D. and Millward, S., Nucleic Acids Research 4 (1977) 3581-3587; Fresno, M., et al., Eur. J. Biochem. 68 (1976) 355-364; Pelham, H. R. and Jackson, R. J., Eur. J. Biochem. 67 (1976) 247-256 and in WO 98/31827.

It has been found that based on the amplification of nucleic acid encoding cognate IgG HC (immunoglobulin G heavy chain) and IgG LC (immunoglobulin G light chain) of an IgG isotype immunoglobulin from a single cell and the subsequent in vitro translation of the obtained nucleic acid to provide Fab fragments of said immunoglobulin a high sensitive method for obtaining information about an immunoglobulin produced by a single cell from the minute amounts of mRNA obtainable is provided. The method according to the invention permits the investigation of the expressed immunoglobulin from a single B-cell, thus, providing higher diversity as opposed to the hybridoma technology. Furthermore, since the cognate immunoglobulin variable domains or immunoglobulin chains are obtained from mature B-cells after antigen contact, selectively the nucleic acid encoding high specific and correctly assembled immunoglobulins can be obtained.

Therefore, one aspect of the current invention is a method for producing an immunoglobulin Fab fragment comprising the following steps:
providing a single immunoglobulin producing cell,
obtaining from the cell the nucleic acid encoding the immunoglobulin light and heavy chain variable domains, optionally also encoding a part of the light chain constant domain and a part of the heavy chain CHI domain,
optionally generating a linear expression matrix comprising the obtained nucleic acid,
translating in vitro the nucleic acid and thereby producing the immunoglobulin Fab fragment.

In one embodiment the nucleic acid encoding the immunoglobulin variable domains is obtained with a method according to the previous aspects of the current invention. In one embodiment of method according to the invention the obtaining the nucleic acid encoding the immunoglobulin light and heavy chain variable domain form a single cell comprises a multiplex polymerase chain reaction according to the invention for the amplification of cognate IgG HC and IgG LC (human IgG isotype) from a single B-cell. For characterization of the binding characteristics of the immunoglobulin encoded by the obtained nucleic acid the nucleic acid is subsequently translated in vitro in an E. coli lysate to an immunoglobulin Fab fragment.

In general one aspect of the current invention is a method employing the following steps i) isolating with magnetic micro-beads coated with the human CD19 B-cells from peripheral blood, ii) depositing single cells by limited dilution or FACS, iii) extracting the mRNA of the individualized B-cells, iv) obtaining the nucleic acid encoding at least the variable domains of the immunoglobulin produced by the individualized B-cell, v) in vitro translating the linear mRNA template, and optionally vi) characterizing the binding properties of the immunoglobulin or immunoglobulin fragment.

For the recombinant production of an immunoglobulin comprising the variable domains obtained from a single cell with a method according to the invention the obtained nucleic acids encoding the variable domain of the light and heavy immunoglobulin chain are further modified. At first the nucleic acid encoding the variable domain is combined with a nucleic acid encoding an immunoglobulin constant region. In one embodiment the nucleic acid encoding the light chain variable domain is combined with a nucleic acid encoding human kappa light chain constant domain of SEQ ID NO: 03 or with a nucleic acid encoding human lambda light chain variable domain of SEQ ID NO: 04. In another embodiment the nucleic acid encoding the heavy chain variable domain is combined with a nucleic acid encoding human immunoglobulin G1 (IgG1) constant region of SEQ ID NO: 01 or with a nucleic acid encoding human immunoglobulin G4 (IgG4) constant region of SEQ ID NO: 02. In another embodiment the nucleic acid encoding the heavy chain variable domain is combined with a nucleic acid encoding human immunoglobulin G1 (IgG1) constant region 1 (C_{H}1).

The nucleic acid molecules encoding the complete immunoglobulin heavy and light chain or a fragment thereof are in the following referred to as structural genes. They can be located on the same expression plasmid or can alternatively be located on different expression plasmids. The assembly of the immunoglobulin or Fab-fragment takes place before the secretion of the immunoglobulin to the cultivation medium and, thus, within the expressing cell. Therefore, the nucleic acid molecules encoding the immunoglobulin chains are in one embodiment expressed in the same host cell. If after recombinant expression a mixture of immunoglobulins is obtained, these can be separated and purified by methods known to a person skilled in the art. These methods are well established and widespread used for immunoglobulin purification and are employed either alone or in combination. Such methods are, for example, affinity chromatography using microbial-derived proteins (e.g. protein A or protein G affinity chromatography), ion exchange chromatography (e.g. cation exchange (carboxymethyl resins), anion exchange (amino ethyl resins) and mixed-mode exchange chromatography), thiophilic adsorption (e.g. with beta-mercaptoethanol and other SH ligands), hydrophobic interaction or aromatic adsorption chromatography (e.g. with phenyl-sepharose, aza-arenophilic resins, or m-aminophenylboronic acid), metal chelate affinity chromatography (e.g. with Ni(II)- and Cu(II)-affinity material), size exclusion chromatography, and preparative electrophoretic methods (such as gel electrophoresis, capillary electrophoresis) (Vijayalakshmi, M. A., Appl. Biochem. Biotech. 75 (1998) 93-102).

With recombinant engineering methods known to a person skilled in the art the conjugates can be tailor-made on the nucleic acid/gene level. The nucleic acid sequences encoding immunoglobulin light and heavy chains are known and can be obtained for example from genomic databases. The elements required for the construction of an expression plasmid for the expression of the immunoglobulin obtained with a method according to the invention are, for example, an expression cassette for the immunoglobulin light chain, an expression cassette for the immunoglobulin heavy chain (alternatively the light chain and the heavy chain structural genes can be contained in the same expression cassette, e.g. as bicistronic expression element), a selection marker, and an E. coli replication as well as selection unit. An expression cassette comprises in general a promoter, a DNA segment encoding secretion signal sequence, the structural gene, and a terminator/polyadenylation signal. The elements are assembled in an operatively linked form either on one plasmid encoding all chains of the immunoglobulin, or on two plasmids each encoding one chain of the immunoglobulin. For the expression of the structural genes the plasmid(s) is (are) introduced into a suitable host cell. Proteins are produced in mammalian cells such as CHO cells, NS0 cells, Sp2/0 cells, COS cells, HEK cells, K562 cells, BHK cells, PER.C6® cells, and the like. In one embodiment the conjugate is expressed in a CHO cell, or a BHK cell, or a HEK cell, or NS0 cell. The regulatory elements of the plasmid have to be selected in a way that they are functional in the selected host cell. For expression the host cell is cultivated under conditions suitable for the expression of the immunoglobulin, which are known to a person of skill in the art. The expressed immunoglobulin chains are functionally assembled and the fully processed immunoglobulin is secreted into the medium.

An “expression plasmid” is a nucleic acid providing all required elements for the expression of the comprised structural gene(s) in a host cell. Typically, an expression plasmid comprises a prokaryotic plasmid propagation unit, e.g. for E. coli, comprising an origin of replication, and a selectable marker, an eukaryotic selection marker, and one or more expression cassettes for the expression of the structural gene(s) of interest each comprising a promoter, a structural gene, and a transcription terminator including a polyadenylation signal. Gene expression is usually placed under the control of a promoter, and such a structural gene is said to be “operably linked to” the promoter. Similarly, a regulatory element and a core promoter are operably linked if the regulatory element modulates the activity of the core promoter.

“Operably linked” refers to a juxtaposition of two or more components, wherein the components so described are in a relationship permitting them to function in their intended manner. The term “linking . . . in operable form” denotes the combination of two or more individual nucleic acids in a way that the individual nucleic acids are operably linked in the final nucleic acid. For example, a promoter and/or enhancer are operably linked to a coding sequence, if it acts in cis to control or modulate the transcription of the linked sequence. Generally, but not necessarily, the DNA sequences that are “operably linked” are contiguous and, where necessary to join two protein encoding regions such as first domain and a second domain, e.g. an immunoglobulin variable domain and an immunoglobbulin constant domain or constant region, contiguous and in (reading) frame. A polyadenylation site is operably linked to a coding sequence if it is located at the downstream end of the coding sequence such that transcription proceeds through the coding sequence into the polyadenylation sequence. A translation stop codon is operably linked to an exonic nucleic acid sequence if it is located at the downstream end (3′ end) of the coding sequence such that translation proceeds through the coding sequence to the stop codon and is terminated there. Linking is accomplished by recombinant methods known in the art, e.g., using PCR methodology and/or by ligation at convenient restriction sites. If convenient restriction sites do not exist, then synthetic oligonucleotide adaptors or linkers are used in accord with conventional practice.

Thus, one aspect of the current invention is a method for producing an immunoglobulin comprising the following steps:
providing a single immunoglobulin producing cell,
obtaining from this cell the nucleic acid encoding the immunoglobulin light and heavy chain variable domains,
linking the nucleic acid encoding the light chain variable domain with a nucleic acid encoding an immunoglobulin light chain constant domain of SEQ ID NO: 03 or SEQ ID NO: 04 in operable form and linking the nucleic acid encoding the heavy chain variable domain with a nucleic acid encoding an immunoglobulin heavy chain constant region of SEQ ID NO: 01 or SEQ ID NO: 02 in operable form,
transfecting a eukaryotic or prokaryotic cell with the nucleic acids of the previous step,
cultivating the transfected cell under conditions suitable for the expression of the immunoglobulin,
recovering the immunoglobulin from the cell or the cultivation medium and thereby producing an immunoglobulin.

The term “under conditions suitable for the expression of” denotes conditions which are used for the cultivation of a cell capable of expressing a heterologous polypeptide and which are known to or can easily be determined by a person skilled in the art. It is known to a person skilled in the art that these conditions may vary depending on the type of cell cultivated and type of polypeptide expressed. In general the cell is cultivated at a temperature, e.g. between 20° C. and 40° C., and for a period of time sufficient to allow effective production of the conjugate, e.g. for of from 4 days to 28 days, in a volume of 0.01 liter to 107 liter.

The following examples, sequence listing and figures are provided to aid the understanding of the present invention, the true scope of which is set forth in the appended claims. It is understood that modifications can be made in the procedures set forth without departing from the spirit of the invention.

### DESCRIPTION OF THE SEQUENCE LISTING

SEQ ID NO: 01 human IgG1 heavy chain constant region
SEQ ID NO: 02 human IgG4 heavy chain constant region
SEQ ID NO: 03 human IgG kappa light chain constant domain
SEQ ID NO: 04 human IgG lambda light chain constant domain
SEQ ID NO: 05 primer V_{H}L-1 variant 1
SEQ ID NO: 06 primer V_{H}L-1 variant 2
SEQ ID NO: 07 primer V_{H}L-2 variant 1
SEQ ID NO: 08 primer V_{H}L-2 variant 2
SEQ ID NO: 09 primer V_{H}L-3
SEQ ID NO: 10 primer V_{H}L-4 variant 1
SEQ ID NO: 11 primer V_{H}L-4 variant 2
SEQ ID NO: 12 primer V_{H}L-5
SEQ ID NO: 13 primer V_{H}L-6
SEQ ID NO: 14 primer huC_{H}-II variant 1
SEQ ID NO: 15 primer huC_{H}-II variant 2
SEQ ID NO: 16 primer VₖL-1
SEQ ID NO: 17 primer VₖL-2
SEQ ID NO: 18 primer VₖL-3
SEQ ID NO: 19 primer VₖL-4
SEQ ID NO: 20 primer huCₖ-II
SEQ ID NO: 21 primer VₗL1
SEQ ID NO: 22 primer VₗL2
SEQ ID NO: 23 primer VₗL3 variant 1
SEQ ID NO: 24 primer VₗL3 variant 2
SEQ ID NO: 25 primer VₗL3 variant 3
SEQ ID NO: 26 primer VₗL3 variant 4
SEQ ID NO: 27 primer huCₗ-II variant 1
SEQ ID NO: 28 primer huCₗ-II variant 2
SEQ ID NO: 29 primer VₗL-1a variant 1
SEQ ID NO: 30 primer V_{H}L-1a variant 2
SEQ ID NO: 31 primer V_{H}L-1b
SEQ ID NO: 32 primer V_{H}L-1c variant 1
SEQ ID NO: 33 primer V_{H}L-1c variant 2
SEQ ID NO: 34 primer V_{H}L-1d variant 1
SEQ ID NO: 35 primer V_{H}L-1d variant 2
SEQ ID NO: 36 primer V_{H}L-2a
SEQ ID NO: 37 primer V_{H}L-2b variant 1
SEQ ID NO: 38 primer V_{H}L-2b variant 2
SEQ ID NO: 39 primer V_{H}L-3a variant 1
SEQ ID NO: 40 primer V_{H}L-3a variant 2
SEQ ID NO: 41 primer V_{H}L-3b
SEQ ID NO: 42 primer V_{H}L-3c
SEQ ID NO: 43 primer V_{H}L-4a variant 1
SEQ ID NO: 44 primer V_{H}L-4a variant 2
SEQ ID NO: 45 primer V_{H}L-4b
SEQ ID NO: 46 primer V_{H}L-5a variant 1
SEQ ID NO: 47 primer V_{H}L-5a variant 2
SEQ ID NO: 48 primer V_{H}L-6a
SEQ ID NO: 49 primer V_{H}L-7a variant 1
SEQ ID NO: 50 primer V_{H}L-7a variant 2
SEQ ID NO: 51 primer huC_{H}-III variant 1
SEQ ID NO: 52 primer huC_{H}-III variant 2
SEQ ID NO: 53 primer VₖL-1a variant 1
SEQ ID NO: 54 primer VₖL-1a variant 2
SEQ ID NO: 55 primer VₖL-1b variant 1
SEQ ID NO: 56 primer VₖL-1b variant 2
SEQ ID NO: 57 primer VₖL-1c variant 1
SEQ ID NO: 58 primer VₖL-1c variant 2
SEQ ID NO: 59 primer VₖL-1d
SEQ ID NO: 60 primer VₗL-2a
SEQ ID NO: 61 primer VₖL-2b variant 1
SEQ ID NO: 62 primer VₖL2b variant 2
SEQ ID NO: 63 primer VₖL3a variant 1
SEQ ID NO: 64 primer VₖL3a variant 2
SEQ ID NO: 65 primer VₖL-3b
SEQ ID NO: 66 primer VₖL-3c
SEQ ID NO: 67 primer VₖL-4a
SEQ ID NO: 68 primer VₖL-5a
SEQ ID NO: 69 primer VₖL-6a
SEQ ID NO: 70 primer VₖL-6b
SEQ ID NO: 71 primer huCₖ-III
SEQ ID NO: 72 primer VₗL-1a
SEQ ID NO: 73 primer VₗL-1b variant 1
SEQ ID NO: 74 primer VₗL-1b variant 2
SEQ ID NO: 75 primer VₗL-1c
SEQ ID NO: 76 primer VₗL-2a
SEQ ID NO: 77 primer VₗL-3a variant 1
SEQ ID NO: 78 primer VₗL-3a variant 2
SEQ ID NO: 79 primer VₗL-3b
SEQ ID NO: 80 primer VₗL-3c
SEQ ID NO: 81 primer VₗL-3d
SEQ ID NO: 82 primer VₗL-4 variant 1
SEQ ID NO: 83 primer VₗL-4 variant 2
SEQ ID NO: 84 primer VₗL-5 variant 1
SEQ ID NO: 85 primer VₗL-5 variant 2
SEQ ID NO: 86 primer VₗL-6
SEQ ID NO: 87 primer VₗL-7 variant 1
SEQ ID NO: 88 primer VₗL-7 variant 2
SEQ ID NO: 89 primer VₗL-8
SEQ ID NO: 90 primer VₗL-4/9 variant 1
SEQ ID NO: 91 primer VₗL-4/9 variant 2
SEQ ID NO: 92 primer VₗL-10
SEQ ID NO: 93 primer huCₗ-III
SEQ ID NO: 94 primer huV_{H}-1
SEQ ID NO: 95 primer huV_{H}-2
SEQ ID NO: 96 primer huV_{H}-3 variant 1
SEQ ID NO: 97 primer huV_{H}-3 variant 2
SEQ ID NO: 98 primer huV_{H}-3 variant 3
SEQ ID NO: 99 primer huV_{H}-3 variant 4
SEQ ID NO: 100 primer huV_{H}-4 variant 1
SEQ ID NO: 101 primer huV_{H}-4 variant 2
SEQ ID NO: 102 primer huV_{H}-4 variant 3
SEQ ID NO: 103 primer huV_{H}-4 variant 4
SEQ ID NO: 104 primer huC_{H}-2 variant 1
SEQ ID NO: 105 primer huC_{H}-2 variant 2
SEQ ID NO: 106 primer huC_{H}-2 variant 3
SEQ ID NO: 107 primer huVₖ-1 variant 1
SEQ ID NO: 108 primer huVₖ-1 variant 2
SEQ ID NO: 109 primer huVₖ-1 variant 3
SEQ ID NO: 110 primer huVₖ-1 variant 4
SEQ ID NO: 111 primer huVₖ-2 variant 1
SEQ ID NO: 112 primer huVₖ-2 variant 2
SEQ ID NO: 113 primer huVₖ-3
SEQ ID NO: 114 primer huVₖ-4
SEQ ID NO: 115 primer huCₖ-2
SEQ ID NO: 116 primer huVₗ-1
SEQ ID NO: 117 primer huVₗ-2
SEQ ID NO: 118 primer huVₗ-3 variant 1
SEQ ID NO: 119 primer huVₗ-3 variant 2
SEQ ID NO: 120 primer huCₗ-2 variant 1
SEQ ID NO: 121 primer huCₗ-2 variant 2
SEQ ID NO: 122 primer huCₗ-2 variant 3
SEQ ID NO: 123 primer huCₗ-2 variant 4
SEQ ID NO: 124 primer huCₗ-2 variant 5
SEQ ID NO: 125 primer huCₗ-2 variant 6
SEQ ID NO: 126 primer LTGS-lfp
SEQ ID NO: 127 primer LTGS-rfp
SEQ ID NO: 128 primer V_{H}-1a
SEQ ID NO: 129 primer V_{H}-1b
SEQ ID NO: 130 primer V_{H}-1c
SEQ ID NO: 131 primer V_{H}-1d
SEQ ID NO: 132 primer V_{H}-2a
SEQ ID NO: 133 primer V_{H}-2b
SEQ ID NO: 134 primer V_{H}-3a
SEQ ID NO: 135 primer V_{H}-3b
SEQ ID NO: 136 primer V_{H}-3c
SEQ ID NO: 137 primer V_{H}-4a
SEQ ID NO: 138 primer V_{H}-4b
SEQ ID NO: 139 primer V_{H}-5a
SEQ ID NO: 140 primer V_{H}-6a
SEQ ID NO: 141 primer V_{H}-7a
SEQ ID NO: 142 primer V_{H}-lfp
SEQ ID NO: 143 primer V_{H}-rfp
SEQ ID NO: 144 primer VL(k)-lfp
SEQ ID NO: 145 primer VL(k)-rfp

### DESCRIPTION OF THE FIGURES

FIGS. 1A-C Chromosomal localization of the human immunoglobulin G heavy chain locus (FIG. 1A), the human immunoglobulin kappa light chain locus (FIG. 1B) and of the human immunoglobulin lambda light chain locus (FIG. 1C).

FIG. 2 Scheme for the polymerase chain reaction for the immunoglobulin light chain with a first and a second primer set.

FIGS. 3A-B Agarose gel analysis of the amplified nucleic acid after the first (FIG. 3A) and the second (FIG. 3B) polymerase chain reaction with different primer sets; (A) 1—IgG HC and IgG LC(κ) 55° C.; 2—IgG LC(κ) 55° C.; 3—IgG HC 55° C.; 4—IgG HC and IgG LC(κ) 50° C.;
5—IgG LC(κ) 50° C.; 6—IgG HC 50° C.; 7—H₂O PCRI; (B)
1—IgG LC(κ) 55° C.; 2—IgG HC and IgG LC(κ) 55° C.; 3—IgG HC 55° C.; 4—IgG
   HC and IgG LC(κ) 50° C.; 5-IgG LC(κ) 50° C.; 6-IgG HC 50° C.;
   7—H₂O PCRII; 8—H₂O PCRI.

FIG. 4 Scheme for the polymerase chain reaction for the immunoglobulin heavy chain with a different second primer set in the two polymerase chain reactions.

FIGS. 5A-B Agarose gel analysis of the amplified nucleic acid after the first (FIG. 5A) and the second (FIG. 5B) polymerase chain reaction with different primer sets; (A) 1—IgG HC and IgG LC(κ) 55° C.; 2—IgG LC(κ) 55° C.; 3—IgG HC 55° C.; 4—IgG HC and IgG LC(κ) 50° C.; 5—IgG LC(κ) 50° C.; 6—IgG HC 50° C.; 7—H₂O PCRI; (B) 1—IgG HC and IgG LC(κ) 55° C.; 2—IgG LC(κ) 55° C.; 3—IgG HC 55° C.; 4—IgG HC und IgG LC(κ) 50° C.; 5-IgG LC(κ) 50° C.; 6—IgG HC 50° C.; 7—H₂O PCRII; 8-H₂O PCRI.

FIG. 6 Scheme for the polymerase chain reaction for the immunoglobulin heavy chain with identical primer set in the two polymerase chain reactions.

FIGS. 7A-B Agarose gel analysis of the amplified nucleic acid after the first (FIG. 7A) and the second (FIG. 7B) polymerase chain reaction with different primer sets; (A) 1—IgG HC and IgG LC(κ) 55° C.; 2—IgG LC(κ) 55° C.; 3—IgG HC 55° C.; 4—IgG HC and IgG LC(κ) 50° C.; 5—IgG LC(κ) 50° C.; 6—IgG HC 50° C.; 7—H₂O PCRI; (B) 1—IgG LC(κ) 55° C.; 2—IgG HC and IgG LC(κ) 55° C.; 3—IgG HC 55° C.; 4—IgG HC and IgG LC(κ) 50° C.; 5—IgG LC(κ) 50° C.; 6—IgG HC 50° C.; 7—H₂O PCRII; 8—H₂O PCRI.

FIG. 8 Scheme of overlapping extension PCR exemplified with a C-terminal HA-tag.

FIG. 9 Agarose gel analysis of the linear expression constructs of the three different polymerase chain reactions of examples 1 to 3.

FIG. 10 Comparison of the result of the three polymerase chain reactions according to the invention after in vitro translation (determination at 450 nm, reference wavelength at 620 nm, background substracted).

FIGS. 11A-B Agarose gel analysis of the amplified nucleic acid after the first (FIG. 11A) and the second (FIG. 11B) polymerase chain reaction with two identical sets of primer; 1—no RT, 2—37 single cells, 38—H₂O cDNA, 39—control mRNA, 40—H₂O control mRNA, 4—H₂O PCRII, 42—IgG HC/IgG LC(κ), 43—IgG HC, 44—IgG LC(κ), 45—H₂O PCRI, 46—GFP, 46—H₂O GFP.

FIGS. 12A-C Agarose gel analysis of the amplified nucleic acid after the second polymerase chain reaction with one variable and one fixed set of primer (FIG. 12A-C).

FIG. 13 Agarose gel analysis of the linear expression constructs obtained from nucleic acid after the second polymerase chain reaction with one variable and one fixed set of primer.

FIG. 14 Result of the in vitro translation of an IgG specific two-step polymerase chain reaction of a single cell with one fixed set of primer and one variable set of primer.

FIG. 15 Human Fab immunoglobulin fragments after single cell polymerase chain reaction and in vitro translation; 1-8 human Fab fragments after in vitro translation of IgG HC and IgG LC(κ) obtained from a single cell and addition of IgG HC control sample, 9-11 human Fab fragments after in vitro translation of IgG HC and IgG LC(κ) obtained from a single cell, 12 human Fab fragments after in vitro translation of IgG HC and IgG LC(λ) obtained from a single cell.

FIGS. 16A-B Western blot analysis after a two-step polymerase chain reaction with one fixed set of primer and one variable set of primer and in vitro translation; (FIG. 16A) 1—IgG HC/IgG LC from a single cell combined with IgG HC, 2—4 IgG HC/IgG LC from a single cell, 5—7 IgG HC/IgG LC(κ) control, 8—IgG HC and IgG LC(κ) control, 9—negative control, 10—standard Fab 0.5 ng/ml, 11—standard Fab 50 ng/ml, 12—standard Fab 5 μg/ml; (FIG. 16B) 1—standard Fab 5 μg/ml, 2—standard Fab 50 ng/ml, 3—standard Fab 0.5 ng/ml, 3—negative control, 5—12 IgG HC/IgG LC from a single cell combined with IgG HC.

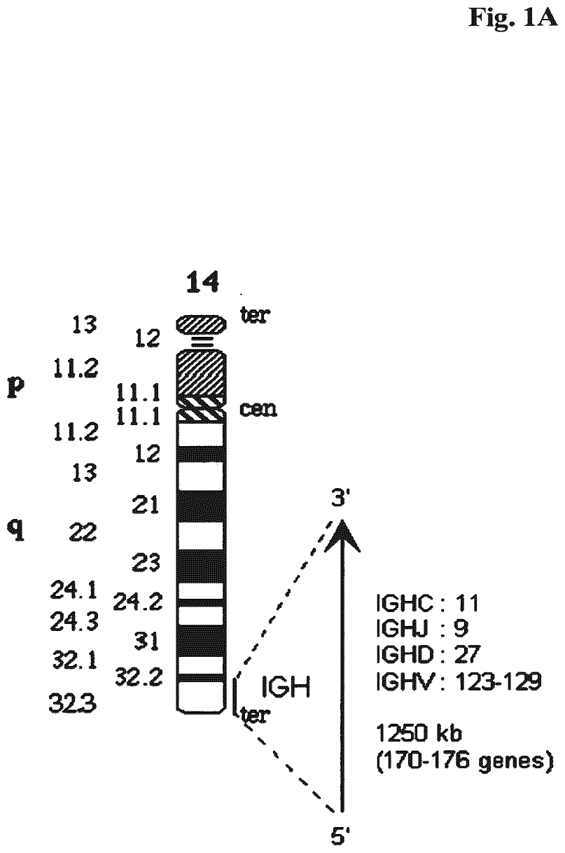

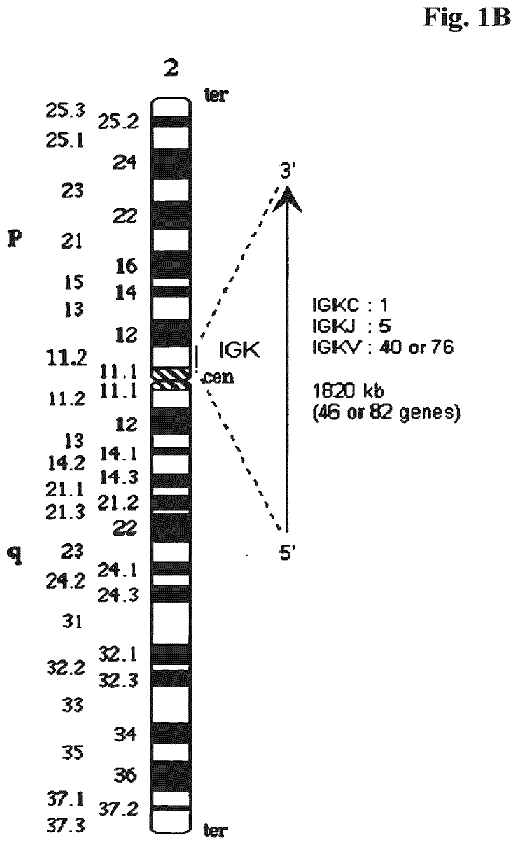

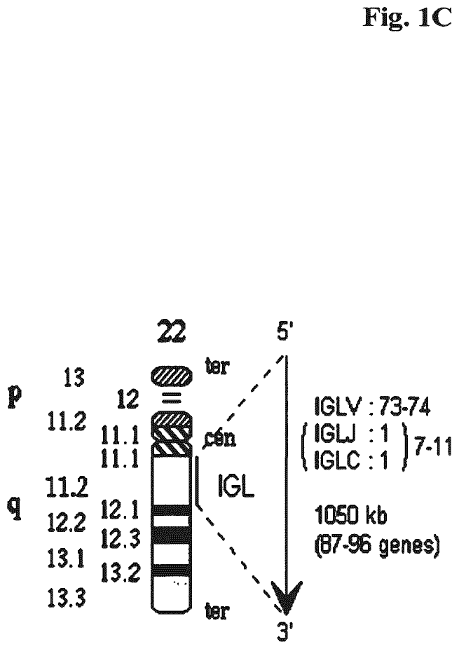

### EXAMPLES

### Materials & Methods

### B-Cells and Plasma Cells

Samples used in this approach are B-cells and plasma cells isolated from the peripheral blood of healthy donor and tissue (spleen, bone marrow) of transgenic mice for human IgG. Solid tissue is first of all manually disaggregated in DMEM in separate tubes. In the later steps, gentle handling and low temperature minimize cell lysis, which is important for the future positive isolation of the cells of interest and to keep the source of mRNA intact. Disaggregated tissue is suspended by the delicate addition of cell separation media for making of a different cell type gradient (Leucosep™-tubes (Greiner Bio-One) with Ficoll® density gradient). Suspended cells are purified by centrifugation on the cold separation medium for 20 min. at 800×g and 22° C. in a centrifuge without breaking in order to enrich for plasma cells (PBMC) and lymphocytes. Cells are washed in cold buffer (PBS (phosphate buffered saline), 0.1% (w/v) BSA (bovine serum albumin), 2 mM EDTA (ethylene diamin tetra acetate)) and the supernatant is carefully discarded to keep only the lymphocytes. Lymphocytes are than resuspended in PBS and mixed by carefully pipetting. Centrifugation is effectuated for 5 min. at 800×g and 22° C. to pellet the cells. B-cells and plasma cells are pretreated with murine and human FC blocker to block unspecific binding of Abs on their cells surface. Cells are washed once with buffer (PBS, 0.1% (w/v) BSA, 2 mM EDTA), centrifuged and resuspended in PBS. Only the CD19+ B-cells and CD138+ plasma cells were used. To prevent mRNA degradation an RNAse Inhibitor is added. The positive isolation of the CD19+ B-cells (Dynal Biotech Dynabeads® magnetic beads CD19 Pan B) from the mouse spleen has been carried out according to the manufacturer's instructions. The selection of the CD138+ plasma cells (StemCell Technologies EasySep™ Human CD138 Selection Kit) has been carried out following the manufacturer's instructions.

### Separation Into Single Cell by the Principle of the Limiting-Dilution Culture or FACS Sorting

Cells are counted and, by the principle of the limiting-dilution culture, deposited as single cell into the wells of 96-well PCR plates or 384-well plates. Plates are sealed with PCR Film and immediately placed on ice. Sorted cells can be used immediately in RT-PCR (reverse transcriptase polymerase chain reaction) or stored at −20° C. for short-term use or −80° C. for long-term use. Single-cell sorting was performed on a FACSAria™ cell-sorting system (Becton Dickinson). Cells that stained positive for CD19, highly positive for CD38 and intermediately positive for CD45 were collected and designated plasma cells (PC). Additional gates on forward scatter/side scatter and side scatter width/side scatter height were included to select live lymphocytes and singlets, respectively. Single cells were distributed directly into the wells of 96-well PCR plates (Eppendorf), containing all the necessary PCR reagents in a volume of 10 μl, except for reverse transcriptase, DNA polymerase, buffer and dNTPs and frozen at −80° C. for later processing.

### Cell Lysis and Reverse Transcription

To be able to amplificate the mRNA in a polymerase chain reaction, B-cells and plasma cells must be lysed before the reverse transcription reaction.

TABLE 22
Components used for the classical lysis.

| Component | Volume (μl) | Final concentration | |
|---|---|---|---|
| Water, PCR grade | 1.75 | | |
| 5xRT Reaction Buffer | 1.00 | | |
| RNAse Inhibitor (40 U/μl) | 0.25 | 5 | U |
| Gene Specific antisense primer | 1.00 | 0.02 | μM |

| | | |
|---|---|---|
| IGEPAL ® detergent 0.01% | 1.00 | 0.01% |
| Final volume | 5.00 | |

TABLE 23
Block cycler program for the cell lysis.

| Temperature (° C.) | Time (sec) |
|---|---|
| 65 | 60 |
| 55 | 30 |
| 45 | 30 |
| 35 | 20 |
| 23 | 120 |
| 4 | ∞ |

Plates with lysed cells are spun briefly in the centrifuge for 30 sec. to collect liquid and cells in the bottom of wells. The RT (reverse transcriptase) reaction as well as all the PCR reaction was made in a 96-well plate. To each well containing 5 μl template is added 2.5 μl of water, 1 μl cold RT reaction buffer, 1 μl dNTPs, 0.25 μl RNAse Inhibitor (40 U/μl), 0.25 μl reverse transcriptase (20 U/μl), all from the First Strand cDNA Synthesis Kit (Roche Diagnostics GmbH, Mannheim, Germany) for a total volume of 10 μl. RT plates are briefly centrifuged and placed from ice to 55° C. for 60 min. (with heated lid), heated to 85° C. for 2 min. (to inactivate the reverse transcriptase), in a block cycler (LightCycler® 480 96-well thermal block cycler, Roche Diagnostics GmbH, Mannheim, Germany). Single-stranded cDNA was stored at −20° C. shortly after the reverse transcription reaction to avoid degradation of the cDNA. A control synthesis reaction was simultaneously performed without cells to test for contamination.

TABLE 24
Components used for the reverse transcription reaction.

| Component | Volume (μl) | Final concentration |
|---|---|---|
| Water, PCR grade | 6.8 | |
| PCR Master | 10 | |
| Semi-nested Primer IgG HC | 0.4 | 0.02 μM |
| Semi-nested Primer IgG LC (k) | 0.4 | 0.02 μM |
| Semi-nested Primer IgG LC (l) | 0.4 | 0.02 μM |
| cDNA template from RT reaction | 2 | |
| Final volume | 20 | |

### Cell Lysis and Reverse Transcription

To be able to amplificate the mRNA in a polymerase chain reaction, B-cells and plasma cells must be lysed before the reverse transcription reaction.

### First PCR

TABLE 25
Block cycler program for the first PCR.

| Temperature (° C.) | Time | Number of cycles |
|---|---|---|
| 95 | 2 min. | 1 |
| 94 | 15 sec. | 35 |
| 55 | 30 sec. | |
| 72 | 1 min. | |
| 72 | 10 min. | 1 |
| 4 | ∞ | |

TABLE 26
Primer used for the first PCR.

| Ig heavy chain | Ig light chain (κ) | Ig light chain (λ) |
|---|---|---|
| primer | primer | primer |
| V_{H}L-1 | V_{κ}L-1 | V_{λ}L-1 |
| V_{H}L-2 | V_{κ}L-2 | V_{λ}L-2 |
| V_{H}L-3 | V_{κ}L-3 | V_{λ}L-3 |
| V_{H}L-4 | V_{κ}L-4 | huC_{λ}-II |
| V_{H}L-5 | huC_{κ}-II | |
| V_{H}L-6 | | |
| huC_{H}-II | | |

### Second PCR

The κ and λ light chains and the heavy chains were subsequently amplified with a second-round PCR according to the following protocol. Using semi-nested primer, the second PCR was performed to increase the amount of cDNA copies and to amplify only from the variable part of the light chain (LC) and heavy chain (HC) to the C_{H}1 region. The HC amplification was performed using 16 primer, the kappa LC using 17 primer, and the lambda LC using 14 primer. The genes were amplified in a total volume of 20 μl using 2 μl of the first PCR product, 10 μl of High Fidelity PCR Master PCR master mix (Roche Diagnostics GmbH, Mannheim, Germany; containing 0.4 μM each dNTPs, double concentrated reaction buffer (with 3 mM MgCl₂), 0.02 μM of each primer, all from the High Fidelity PCR Master Kit (Roche Diagnostics GmbH, Mannheim, Germany) using the following second PCR program: 2 min. at 95° C., 45 cycles of 94° C. for 15 sec., 55° C. for 30 sec., 72° C. for 1 min., following 10 min. at 72° C.

TABLE 27
Components used for the second PCR.

| Component | Volume (μl) | Final concentration |
|---|---|---|
| Water, PCR grade | 7.8 | |
| High Fidelity PCR Master | 10 | 1.8 mM MgCl₂ |
| Two-step Primer IgG HC | 0.4 | 0.02 μM |
| Two-step Primer IgG LC(κ) | 0.4 | 0.02 μM |
| Two-step Primer IgG LC(λ) | 0.4 | 0.02 μM |
| First PCR product | 2 | |
| Final volume | 20 | |

TABLE 28
Block cycler program for the second PCR.

| Temperature (° C.) | Time | Number of cycles |
|---|---|---|
| 95 | 2 min. | 1 |
| 94 | 15 sec. | 45 |
| 55 | 30 sec. | |
| 72 | 1 min. | |
| 72 | 10 min. | 1 |
| 4 | ∞ | |

TABLE 29
Primer used for the second PCR

| IgG heavy chain | IgG light chain (κ) | IgG light chain (λ) |
|---|---|---|
| primer | primer | primer |
| V_{H}1a | V_{κ}1a | V_{λ}1a |
| V_{H}1b | V_{κ}1b | V_{λ}1b |
| V_{H}1c | V_{κ}1c | V_{λ}1c |
| V_{H}1d | V_{κ}1d | V_{λ}2 |
| V_{H}2a | V_{κ}2a | V_{λ}3a |
| V_{H}2b | V_{κ}2b | V_{λ}3b |
| V_{H}3a | V_{κ}3a | V_{λ}3c |
| V_{H}3b | V_{κ}3b | V_{λ}3d |
| V_{H}3c | V_{κ}3c | V_{λ}4 |
| V_{H}4a | V_{κ}4a | V_{λ}5 |
| V_{H}4b | V_{κ}5a | V_{λ}6 |
| V_{H}5a | V_{κ}6a | V_{λ}7 |
| V_{H}6a | V_{κ}6b | V_{λ}8 |
| V_{H}7a | C_{κ}III | V_{λ}4/9 |
| C_{H}III | | V_{λ}10 |
| | | C_{λ}III |

### One Step Multiplex RT-GSP (Gene Specific Primer)-PCR Reaction

To be able to amplificate the mRNA in a polymerase chain reaction, B-cells and plasma cells must be distributed directly into the wells of 96-well PCR plates (Eppendorf), containing all the necessary PCR reagents in a volume of 10 μl, except for reverse transcriptase, DNA polymerase, buffer and dNTPs and frozen at −80° C. for later processing.

### RT-Step

Reverse transcription and PCR were performed in one step (one step Multiplex RT-PCR). The isolated, sorted and stored cells were used as raw material for the reverse transcription or RT-PCR. All necessary reagents were thawed at room temperature. All primer were synthesized in the MOLBIOL TIB GmbH laboratories. The plates and all other reagents were kept on ice during the entire procedure. For cDNA syntheses the gene specific primer with extensions were used directly. The enzyme complex consists of two Sensiscript reverse transcriptases and one Omniscript® polymerase (QIAGEN OneStep RT-PCR kit). The rewriting of the mRNA into cDNA was performed by the Sensiscript® complex (QIAGEN OneStep RT-PCR kit) and the amplification of the cDNA was performed using the HotStarTaq® DNA Polymerase (QIAGEN OneStep RT-PCR kit), which is a chemically form of a recombinant 94 kDa DNA polymerase (deoxynucleoside-triphosphate: DNA deoxynucleotidyltransferase, EC 2.7.7.7), originally isolated from Thermus aquaticus expressed in E. coli. The cells were sorted in a 96-well PCR plate and stored in a volume of 10 μl, containing 5 μl PCR H₂O grade, 1 μl 0.1 μM primer for VH and VL, 1 μl RNAse inhibitor 20 U/reaction and 3 μl Tris 1.5 mM. Before adding the other 10 μl for performing the PCR reaction, the cells stored at −60° C. were briefly centrifuged (20 sec. at 1400 rpm) to collect the liquid and cells on the bottom of the wells.

TABLE 30
Master Mix 1 used for the RT-PCR.

| | Final | volume/well |
|---|---|---|
| Master Mix 1 | concentration/well | (μl) |
| H₂O | | 5 |
| primer V_{H}/VL(k) | 0.1 μM | 1 |
| RNAse Inhibitor | 20 U/reaction | 1 |
| Tris-buffer | 1.5 mM | 3 |
| B/Plasma cells | | |
| final volume | | 10 |

TABLE 31
Master Mix 2 used for the RT-PCR.

| | Final | volume/well |
|---|---|---|
| Master Mix 2 | concentration/well | (μl) |
| H₂O | 1× | 2.2 |
| 5× Buffer | 1× | 4 |
| dNTP 10 mM each | 400 μM each | 0.8 |
| 5× Q-Solution | 0.25× | 1 |
| QIAGEN One Step RT PCR | | 1.2 |
| Enzyme mix | | |
| RNAse Inhibitor | 20U | 1 |
| final volume | | 10 |

10 μl per well of Master Mix 2 were added to the cells. The second Master Mix contained 2.2 μl H₂O PCR grade, 4 μl of 1× buffer, 0.8 μl of dNTPs 400 μM each, 1 μl of Q-solution 0.25×, 1.2 μl of the enzyme complex and 1 μl of RNAse inhibitor 20 U.

TABLE 32
Primer used for the RT-PCR.

| Ig heavy chain primer | | Ig light chain (κ) primer | |
|---|---|---|---|
| V_{H}-lfp | SEQ ID NO: 142 | VL(k)-lfp | SEQ ID NO: 144 |
| V_{H}-rfp | SEQ ID NO: 143 | VL(k)-rfp | SEQ ID NO: 145 |

TABLE 33
Block cycler program for the RT-GSP-PCR.

| Temperature | Time | Step | Cycles |
|---|---|---|---|
| 50° C. | 30 min. | reverse transcription | 1 |
| 95° C. | 15 min. | denaturation | 1 |
| 94° C. | 40 sec. | denaturation | 11 |
| 52° C. | 1 min. | annealing | |
| 72° C. | 1 min. | elongation | |
| 94° C. | 41 sec. | denaturation | 29 |
| 60° C. | 1 min. | annealing | |
| 72° C. | 1 min. | elongation | |
| 72° C. | 10 min. | final elongation | 1 |
| 4° C. | ∞ | cooling | |

### Purification of PCR Products

To improve the efficiency of the generation of linear template for the in vitro translation in the next overlapping PCR (third PCR) the purification of the previously amplified PCR products was performed by removing unincorporated primer, dNTPs, DNA polymerases and salts used during PCR amplification in order to avoid interference in downstream applications. Agencourt AMPURE® PCR purification system (Beckman Coulter) was used. The buffer is optimized to selectively bind PCR amplicons 100 bp and larger to paramagnetic beads. Excess oligonucleotides, nucleotides, salts, and enzymes can be removed using a simple washing procedure. The resulting purified PCR product is essentially free of contaminants and can be used in the following applications: Fluorescent DNA sequencing (including capillary electrophoresis), microarray spotting, cloning and primer extension genotyping. The work flow for 96-well format started with gently shaking the beads stored in buffer to resuspend any magnetic particle that may have settled. The correct volume of 36 μl of beads solution was added to the 20 μl of sample and the mix was pipetted 10 times up and down. The following step was incubating for 10 minutes and afterwards the reaction plate was placed onto a magnetic plate for 10 minutes to separate beads from solution. The cleared solution (supernatant) was aspirated from the reaction plate and discard. For the beads-cDNA washing 200 μl of 70% ethanol were dispersed per well and incubated at room temperature for at least 30 seconds. The ethanol was aspirated out and discarded. The washing step was performed two times and then the reaction plate was left to air-dry for 20 minutes at room temperature. It followed with the addition of 40 μl of elution buffer and the mix was again pipetted 10 times up and down. After the cDNA dissociation from the magnetic beads, the purified DNA was transferred into a new plate.

### Third PCR

The amplified DNA of the second PCR was afterwards linked by an overlapping extension PCR method with the following components, necessary for the transcription/translation step: a ribosome binding site (RBS), a T7 promoter and a T7 terminator sequences. For this PCR, 2 μl of the second PCR were taken to a final volume of 20 μl containing: 10.7 μl water, 2 μl of 10× reaction buffer with MgCl₂ (10 mM), 0.8 μl of DMSO, 0.5 μl dNTPs (10 mM each), 1.6 μl T7 promotor and terminator primer (6 μM each), 0.4 μl C-terminal HA-Tag primer and 0.4 μl of enzyme blend, all from the Rapid Translation System E. coli Linear Template Generation Set, HA-Tag (Roche Diagnostics GmbH, Mannheim, Germany). Finally, the overlapping PCR products were used as template for in vitro transcription using Escherichia coli lysate and the resulting functional Fab was screened against the F(ab′)₂ IgG by enzyme-linked immunoadsorbent assay (ELISA).

TABLE 34
Components used for the third PCR.

| | Volume | |
|---|---|---|
| Component | (μl) | Final concentration |
| Water, PCR grade | 10.7 | |
| 10× Reaction Buffer with MgCl₂ (10 mM) | 2 | 1× |
| DMSO | 0.8 | |
| PCR Nucleotide mix (10 mM each) | 0.5 | 250 μM |
| Working solution T7 Prom Primer (6 μM) | 1.6 | 0.48 μM |
| Working solution T7 Term Primer (6 μM) | 1.6 | 0.48 μM |
| Working solution C-term HA-tag (6 μM) | 0.4 | 0.48 μM |
| Enzyme Blend | 0.4 | |
| PCR 2 product | 2 | |
| Final volume | 20 | |

TABLE 35
Block cycler program for the third PCR.

| Temperature (° C.) | Time | Number of cycles |
|---|---|---|
| 95 | 4 min. | 1 |
| 95 | 1 min. | 45 |
| 60 | 1 min. | |
| 72 | 1 min. 30 sec. | |
| 72 | 7 min. | 1 |
| 4 | ∞ | |

### Gel Electrophoresis

The gel electrophoresis analysis (1% agarose gel, Invitrogen Corp., USA) was performed to evaluate the amplification and the specificity of the cDNA templates with the appropriate controls.

TABLE 36
Gel analysis protocol.

| Component | Volume (μl) | Migration time |
|---|---|---|
| H₂0 | 6 | |
| 5× Orange G | 3 | |
| PCR product | 6 | |
| Final volume | 15 | |
| Volume for gel | 10 | 20 min. |

In vitro Transcription and Translation

The in vitro coupled transcription and translation was carried out following the manufacturer's protocol RTS™ 100 E. coli Disulfide Kit (biotechrabbit GmbH, Berlin, Germany) with components as reported (see Table 12). 4 μl of each overlapping PCR product was transcribed and translated in a total volume of 50 μl, at 37° C. for 20 hours in the RTS Proteo Master Instrument (Roche Diagnostics GmbH, Mannheim, Germany). A control reaction was performed under identical conditions without cDNA template. GFP (green fluorescent protein) vectors were added to the reaction system for autoradiography as positive control. After the in vitro transcription/translation, the 50 μl reaction mixture was transferred in 75 μl PBS (1:2.5 dilution) and incubated at 4° C. overnight for the correct folding and maturation of the protein.

TABLE 37
Components for the in vitro transcription and translation.

| Mix | Component | Volume (μl) |
|---|---|---|
| Mix 1: | E. coli lysate | 25 |
| | Lysate activator | 1 |
| | Final volume | 26 |
| | incubate for 10-20 min. at RT | |
| Mix 2: | Feeding mix | 640 |
| | Amino acid mix | 140 |
| | Methionine | 20 |
| | H₂O | 200 |
| | Final volume | 1000 |
| Mix 3: | Reaction mix | 7 |
| | Amino acid mix | 7 |
| | Methionine | 1 |
| | Mix 1 | 25 |
| | GroE Supplement | 5 |
| | RNAse inhibitor | 1 |
| | PCR 3 product | 4 |
| | Final volume | 50 |

### ELISA

A 384-well plate (Nunc GmbH & Co. KG, Thermo Fisher Scientific, Langenselbold, Germany) was coated with 50 μl (1:1000 in PBS) goat anti-human IgG Fab fragment (produced by Bethyl Laboratories Inc., obtained from Biomol GmbH, Hamburg, Germany, 1 mg/1 ml) incubated at 4° C. overnight. The plate was washed three times with washing solution (100 μl PBST (phosphate buffered saline TWEEN®)) and 60 μl of Blocking solution (0.25% CroteinC (w/v)/0.5% Tween (w/v)/PBS) was added, incubated for 1 h at room temperature. Another washing step (3×100 μl PBST) was performed and 37.5 μl sample was transferred, as well as 37.5 ml negative control (negative control from the in vitro transcription/translation) and 37.5 μl positive control, containing 0.75 μl of human recombinant Fab fragment (Roche Diagnostics GmbH, Mannheim, Germany). The samples were titrated to a 1:3 dilution. The plate was incubated for 1.5 h at room temperature. After a washing step (3×100 μl PBST), 25 μl goat anti-human IgG F(ab′)₂ (Dianova, Hamburg, Germany; 0.8 mg/ml (1:2000 diluted in Blocking Solution)) was added and incubated for 1 h at room temperature. The last washing step (3×100 μl PBST) was performed and 25 μl of TMB (POD Substrate, Roche Diagnostics GmbH, Mannheim, Germany, Art-No: 1 484 281) was pipetted into each well. After 2-3 minutes the absorption signal was detected at 405 nm and 495 nm (Safire²™2 microplate reader; Tecan Deutschland GmbH, Crailsheim, Germany).

### Flow Cytometric Analysis and Cell Sorting

For FACS analysis and cell sorting monoclonal antibodies, either biotinylated or conjugated with either FITC (fluorescein isothiocyanate), PE (Phycoerythrin), or APC (allophycocyanin) against the following antigens were used: CD3 (UCHT1), CD4 (13B8.2), CD8 (B9.11), CD40 (MAB89), CD80 (MAB104), CD83 (HB15a), CD86 (HA5.2B7) (all available from Imunotech/Beckman Coulter, Marseille, France), CD19 (HIB19), CD20 (2H7), CD34 (581), IL-3Ra/CD123 (9F5), CD11c (B-ly6) CD14 (M5E2), CD24, CD22a, CD38, CD138 (all available from BD Pharmingen, San Diego, CA, USA), CD45 (HI30), CD45RA (MEM56), HLA-DR (TU36) (all available from Caltag Laboratories, Burlingame, CA, USA), TLR2 (TL2.1), TLRR4 (HTA125), TCRab (IP26), (all available from Bioscience, San Diego, CA), BDCA-1, BDCA-2, BDCA-4, CD25 (4E3) (all available from Miltenyi Biotec, Bergisch Gladbach, Germany), IgM (Jackson Immunoresearch Laboratories, Inc., West Grove, PA, USA), CCR7 (3D12, provided by M. Lipp, Berlin, Germany). The IOTest® Beta Mark TCR VB repertoire kit was used for Vb analysis (Beckman Coulter Immunotech S.A.S, Marseille France). Streptavidin conjugated FITC, PE, or APC (all BD Pharmingen™, San Diego, CA, USA) were used for visualization of biotinylated antibodies. Dead cells were excluded by propidium iodide staining. Appropriate isotype-matched, irrelevant control mAbs were used to determine the level of background staining. Cells were analyzed using a FACS Calibur and sorted using a BD FACSAria™ cell sorter (Becton Dickinson Immunocytometry Systems, Mountain View, CA, USA).

### Example 1

### Amplification of IgG Genes From Humanized Immunized Mice'S Single B Cell by a Polymerase Chain Reaction With One Fixed Primer Set and One Changed Primer Set

Single B-cells of a mouse having a human immunoglobulin locus have been obtained as outlined above. The 3′-primer of the first and second primer set employed in the polymerase chain reaction were identical. The 5′-primer of the first and second primer set different insofar as the primer of the first 5′-primer set bound in the region encoding the leader peptide and the primer of the second

5′-primer set bound in the FRI region. A scheme of this polymerase chain reaction is given in FIG. 2.

The employed sets of primer for the first polymerase chain reaction are denoted in the following list:

| Ig heavy chain | Ig light chain (κ) | |
|---|---|---|
| primer | primer | Ig light chain (λ) primer |
| VHL-1 | V_{κ}L-1 | V_{λ}L-1 |
| V_{H}L-2 | V_{κ}L-2 | V_{λ}L-2 |
| V_{H}L-3 | V_{κ}L-3 | V_{λ}L-3 |
| V_{H}L-4 | V_{κ}L-4 | huC_{λ}-II |
| V_{H}L-5 | huC_{κ}-II | |
| V_{H}L-6 | | |
| huC_{H}-II | | |

The employed sets of primer for the second polymerase chain reaction are denoted in the following list:

| IgG heavy chain | | IgG light chain (κ) | | IgG light chain | |
|---|---|---|---|---|---|
| primer | | primer | | (λ) primer | |
| V_{H}-1a | SEQ ID NO: | V_{κ}L-1a | SEQ ID NO: | V_{λ}L-1a | SEQ ID NO: |
| | 128 | | 53, 54 | | 72 |
| V_{H}-1b | SEQ ID NO: | V_{κ}L-1b | SEQ ID NO: | V_{λ}L-1b | SEQ ID NO: |
| | 129 | | 55, 56 | | 73, 74 |
| V_{H}-1c | SEQ ID NO: | V_{κ}L-1c | SEQ ID NO: | V_{λ}L-1c | SEQ ID NO: |
| | 130 | | 57, 58 | | 75 |
| V_{H}-1d | SEQ ID NO: | V_{κ}L-1d | SEQ ID NO: | V_{λ}L-2 | SEQ ID NO: |
| | 131 | | 59 | | 76 |
| V_{H}-2a | SEQ ID NO: | V_{κ}L-2a | SEQ ID NO: | V_{λ}L-3a | SEQ ID NO: |
| | 132 | | 60 | | 77, 78 |
| V_{H}-2b | SEQ ID NO: | V_{κ}L-2b | SEQ ID NO: | V_{λ}L-3b | SEQ ID NO: |
| | 133 | | 61, 62 | | 79 |
| V_{H}-3a | SEQ ID NO: | V_{κ}L-3a | SEQ ID NO: | V_{λ}L-3c | SEQ ID NO: |
| | 134 | | 63, 64 | | 80 |
| V_{H}-3b | SEQ ID NO: | V_{κ}L-3b | SEQ ID NO: | V_{λ}L-3d | SEQ ID NO: |
| | 135 | | 65 | | 81 |
| V_{H}-3c | SEQ ID NO: | V_{κ}L-3c | SEQ ID NO: | V_{λ}L-4 | SEQ ID NO: |
| | 136 | | 66 | | 82, 83 |
| V_{H}-4a | SEQ ID NO: | V_{κ}L-4a | SEQ ID NO: | V_{λ}L-5 | SEQ ID NO: |
| | 137 | | 67 | | 84, 85 |
| V_{H}-4b | SEQ ID NO: | V_{κ}L-5a | SEQ ID NO: | V_{λ}L-6 | SEQ ID NO: |
| | 138 | | 68 | | 86 |
| V_{H}-5a | SEQ ID NO: | V_{κ}L-6a | SEQ ID NO: | V_{λ}L-7 | SEQ ID NO: |
| | 139 | | 69 | | 87, 88 |
| V_{H}-6a | SEQ ID NO: | V_{κ}L-6b | SEQ ID NO: | V_{λ}L-8 | SEQ ID NO: |
| | 140 | | 70 | | 89 |
| V_{H}-7a | SEQ ID NO: | huC_{κ}-2 | SEQ ID NO: | V_{λ}L-4/9 | SEQ ID NO: |
| | 141 | | 115 | | 90, 91 |
| huC_{H}-2 | SEQ ID NO: | | | V_{λ}L-10 | SEQ ID NO: |
| | 104, 105, | | | | 92 |
| | 106 | | | | |
| | | | | huC_{λ}-2 | SEQ ID NO: |
| | | | | | 120, 121, 122, |
| | | | | | 123, 124, 125 |

In FIGS. 3A-B the agarose gel of the amplified nucleic acid fragments obtained in this polymerase chain reaction is shown. The samples were analyzed after 40 amplification cycles with an annealing temperature of 50° C. and 55° C., respectively. The blanks (water) were negative and the size of the fragments correlated well to the expected sizes of 750 bp (IgG HC) and 665 bp (IgG LC), respectively, after the first polymerase chain reaction and of 711 bp and 688 bp, respectively, after the second polymerase chain reaction.

### Example 2

### Amplification of IgG Genes From Humanized Immunized Mice'S Single B Cell by a Polymerase Chain Reaction With Two Changed Primer Sets

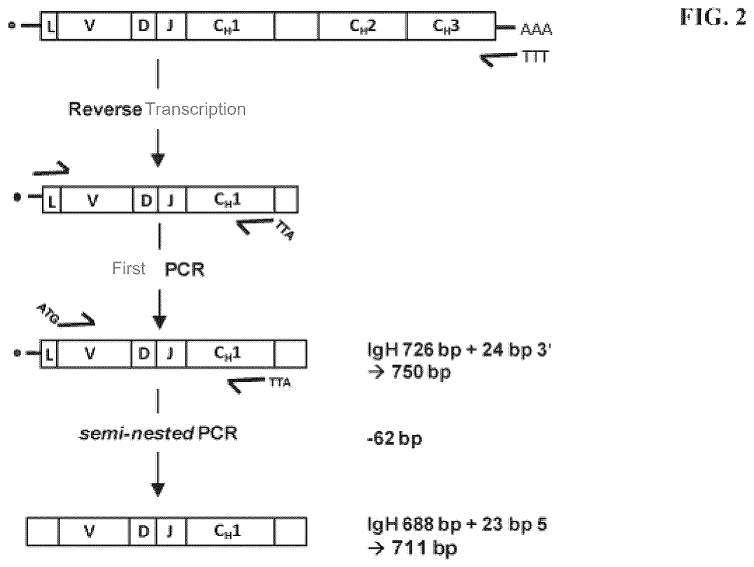

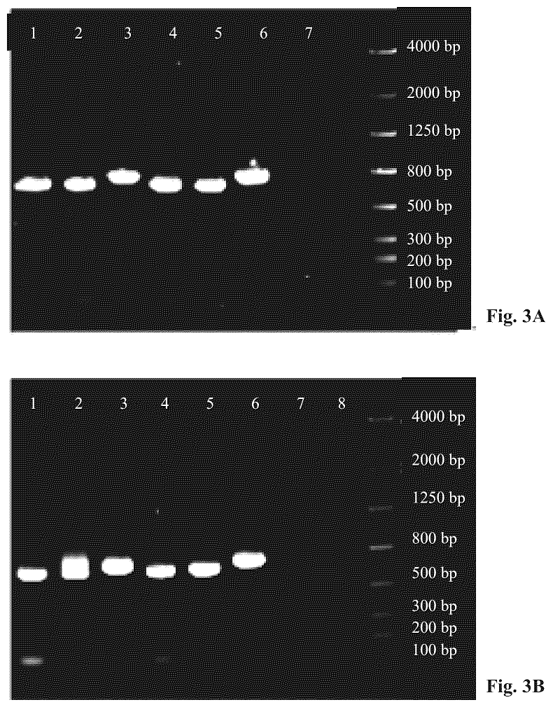

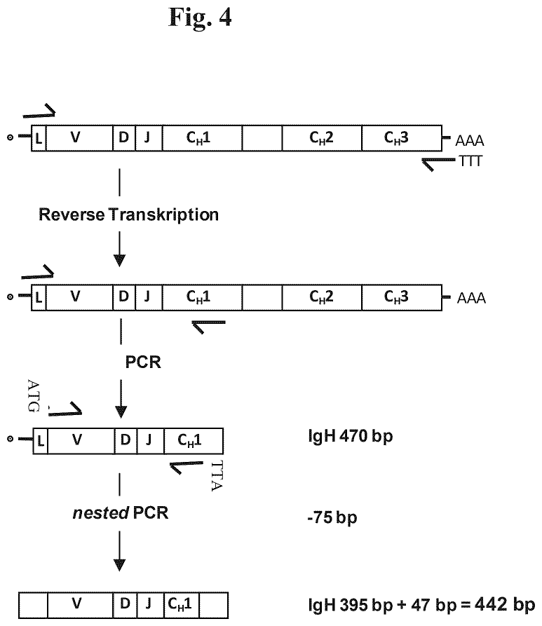

Single B-cells of a mouse having a human immunoglobulin locus have been obtained as outlined above. The 5′-primer and the 3′-primer of the first and second primer set were different to each other insofar as the primer of each of the second sets bound in a more inward region of the nucleic acid. A scheme of this polymerase chain reaction is given in FIG. 4.

The employed sets of primer for the first polymerase chain reaction are denoted in the following list:

| | | Ig light | | | |
|---|---|---|---|---|---|
| Ig heavy chain primer | | chain (κ) primer | | Ig light chain (λ) primer | |
| V_{H}L-1 | SEQ ID NO: | V_{κ}L-1 | SEQ ID NO: | V_{λ}L-1 | SEQ ID NO: |
| | 05, 06 | | 16 | | 21 |
| V_{H}L-2 | SEQ ID NO: | V_{κ}L-2 | SEQ ID NO: | V_{λ}L-2 | SEQ ID NO: |
| | 07, 08 | | 17 | | 22 |
| V_{H}L-3 | SEQ ID NO: | V_{κ}L-3 | SEQ ID NO: | V_{λ}L-3 | SEQ ID NO: |
| | 09 | | 18 | | 23, 24, 25, 26 |
| V_{H}L-4 | SEQ ID NO: | V_{κ}L-4 | SEQ ID NO: | huC_{λ}-II | SEQ ID NO: |
| | 10, 11 | | 19 | | 27, 28 |
| V_{H}L-5 | SEQ ID NO: | huC_{κ}-II | SEQ ID NO: | | |
| | 12 | | 20 | | |
| V_{H}L-6 | SEQ ID NO: | | | | |
| | 13 | | | | |
| huC_{H}-II | SEQ ID NO: | | | | |
| | 14, 15 | | | | |

The employed sets of primer for the second polymerase chain reaction are denoted in the following list:

| IgG heavy | | IgG light chain (κ) | | IgG light | |
|---|---|---|---|---|---|
| chain primer | | primer | | chain (λ) primer | |
| V_{H}L-1a | SEQ ID NO: | V_{κ}L-1a | SEQ ID NO: | V_{λ}L-1a | SEQ ID NO: |
| | 29, 30 | | 53, 54 | | 72 |
| V_{H}L-1b | SEQ ID NO: | V_{κ}L-1b | SEQ ID NO: | V_{λ}L-1b | SEQ ID NO: |
| | 31 | | ?55, 56 | | 73, 74 |
| V_{H}L-1c | SEQ ID NO: | V_{κ}L-1c | SEQ ID NO: | V_{λ}L-1c | SEQ ID NO: |
| | 32, 33 | | 57, 58 | | 75 |
| V_{H}L-1d | SEQ ID NO: | V_{κ}L-1d | SEQ ID NO: | V_{λ}L-2a | SEQ ID NO: |
| | 34, 35 | | 59 | | 76 |
| V_{H}L-2a | SEQ ID NO: | V_{κ}L-2a | SEQ ID NO: | V_{λ}L-3a | SEQ ID NO: |
| | 36 | | 60 | | 77, 78 |
| V_{H}L-2b | SEQ ID NO: | V_{κ}L-2b | SEQ ID NO: | V_{λ}L-3b | SEQ ID NO: |
| | 37, 38 | | 61, 62 | | 79 |
| V_{H}L-3a | SEQ ID NO: | V_{κ}L-3a | SEQ ID NO: | V_{λ}L-3c | SEQ ID NO: |
| | 39, 40 | | 63, 64 | | 80 |
| V_{H}L-3b | SEQ ID NO: | V_{κ}L-3b | SEQ ID NO: | V_{λ}L-3d | SEQ ID NO: |
| | 41 | | 65 | | 81 |
| V_{H}L-3c | SEQ ID NO: | V_{κ}L-3c | SEQ ID NO: | V_{λ}L-4a | SEQ ID NO: |
| | 42 | | 66 | | 82, 83 |
| V_{H}L-4a | SEQ ID NO: | V_{κ}L-4a | SEQ ID NO: | V_{λ}L-5a | SEQ ID NO: |
| | 43, 44 | | 67 | | 84, 85 |
| V_{H}L-4b | SEQ ID NO: | V_{κ}L-5a | SEQ ID NO: | V_{λ}L-6a | SEQ ID NO: |
| | 45 | | 68 | | 86 |
| V_{H}L-5a | SEQ ID NO: | V_{κ}L-6a | SEQ ID NO: | V_{λ}L-7a | SEQ ID NO: |
| | 46, 47 | | 69 | | 87, 88 |
| V_{H}L-6a | SEQ ID NO: | V_{κ}L-6b | SEQ ID NO: | V_{λ}L-8a | SEQ ID NO: |
| | 48 | | 70 | | 89 |
| V_{H}L-7a | SEQ ID NO: | huC_{κ}-III | SEQ ID NO: | V_{λ}L-4/9a | SEQ ID NO: |
| | 49, 50 | | 71 | | 90, 91 |
| huC_{H}- | SEQ ID NO: | | | V_{λ}L-10a | SEQ ID NO: |
| III | 51, 52 | | | | 92 |
| | | | | huC_{λ}-III | SEQ ID NO: |
| | | | | | 93 |

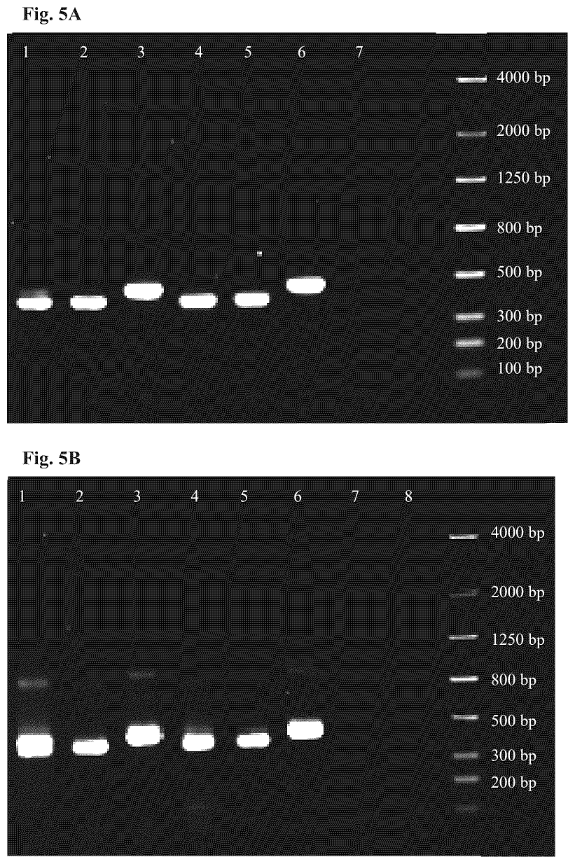

In FIGS. 5A-B the agarose gel of the amplified nucleic acid fragments obtained in this polymerase chain reaction is shown. The samples were analyzed after 40 amplification cycles with an annealing temperature of 50° C. and 55° C., respectively. The blanks (water) were negative and the size of the fragments correlated well to the expected sizes of 471 bp (IgG HC) and 413 bp (IgG LC), respectively, after the first polymerase chain reaction and of 442 bp and 399 bp, respectively, after the second polymerase chain reaction.

### Example 3

### Amplification of IgG Genes From Humanized Immunized Mice'S Single B Cell ay a Polymerase Chain Reaction With Two Identical Primer Sets

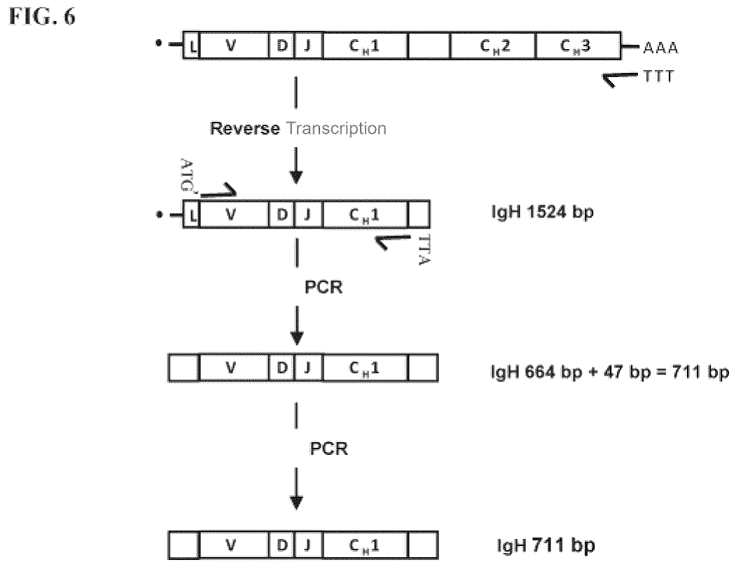

Single B-cells of a mouse having a human immunoglobulin locus have been obtained as outlined above. The 5′-primer and the 3′-primer of the first and second primer set were identical. A scheme of this polymerase chain reaction is given in FIG. 6.

The employed sets of primer for the first and second polymerase chain reaction are denoted in the following list:

| | | Ig light chain (κ) | | | |
|---|---|---|---|---|---|
| Ig heavy chain primer | | primer | | Ig light chain (λ) primer | |
| huV_{H}-1 | SEQ ID NO: | huV_{κ}-1 | SEQ ID NO: | huV_{λ}-1 | SEQ ID NO: |
| | 94 | | 107, 108, | | 116 |
| | | | 109, 110 | | |
| huV_{H}-2 | SEQ ID NO: | huV_{κ}-2 | SEQ ID NO: | huV_{λ}-2 | SEQ ID NO: |
| | 95 | | 111, 112 | | 117 |
| huV_{H}-3 | SEQ ID NO: | huV_{κ}-3 | SEQ ID NO: | huV_{λ}-3 | SEQ ID NO: |
| | 96, 97, 98, | | 113 | | 118, 119 |
| | 99 | | | | |
| huV_{H}-4 | SEQ ID NO: | huV_{κ}-4 | SEQ ID NO: | huC_{λ}-2 | SEQ ID NO: |
| | 100, 101, | | 114 | | 120, 121, 122, |
| | 102, 103 | | | | 123, 124, 125 |
| huC_{H}-2 | SEQ ID NO: | huC_{κ}-2 | SEQ ID NO: | | |
| | 104, 105, | | 115 | | |
| | 106 | | | | |

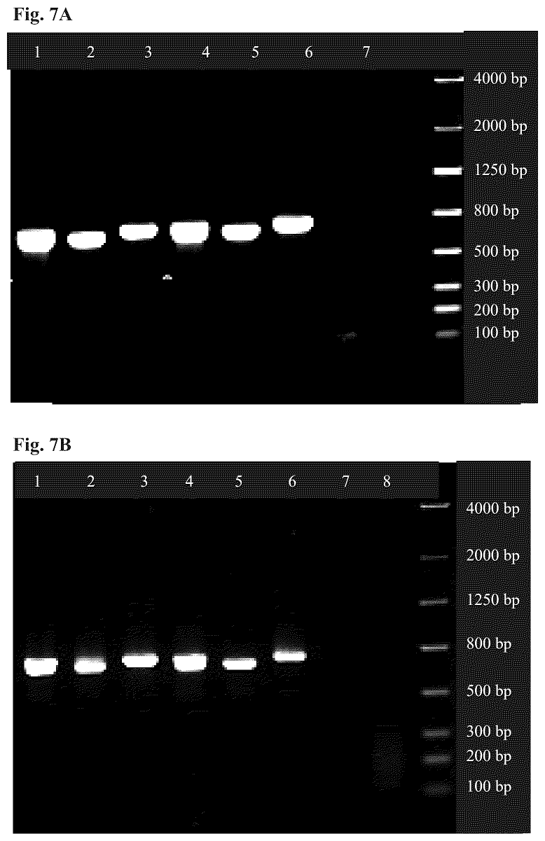

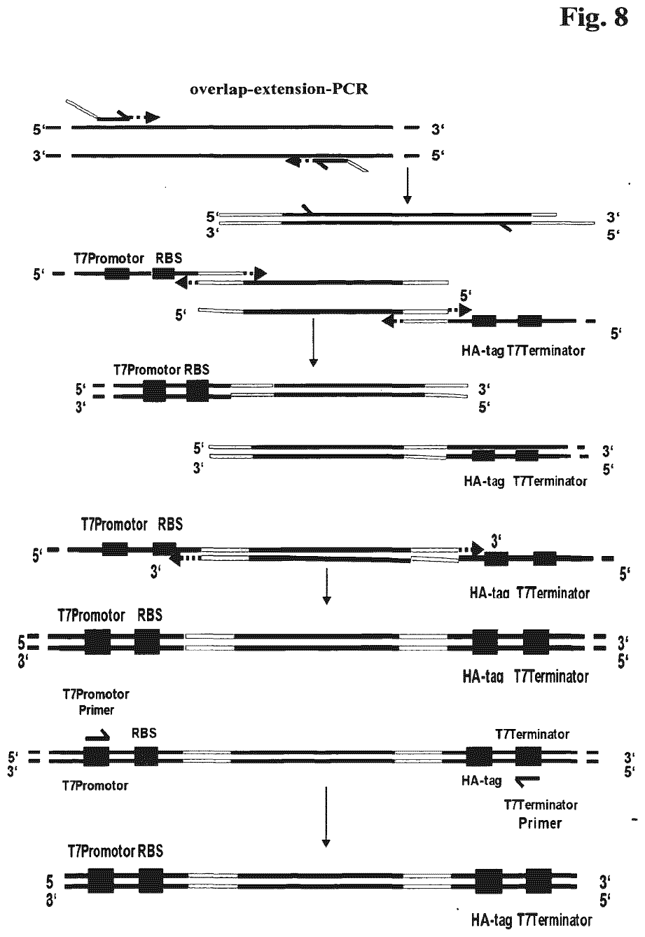

In FIGS. 7A-B the agarose gel of the amplified nucleic acid fragments obtained in this polymerase chain reaction is shown. The samples were analyzed after 40 amplification cycles with an annealing temperature of 50° C. and 55° C., respectively. The blanks (water) were negative and the size of the fragments correlated well to the expected sizes of 711 bp (IgG HC) and 688 bp (IgG LC), respectively, after the first and second polymerase chain reaction.

### Example 4

Generation of Linear Template for in vitro Translation

For the first polymerase chain reaction gene specific primer have been designed comprising the necessary overlapping sequences to the regulatory DNA regions of the T7 phage. For the second polymerase chain reaction the product of the first PCR was combined with nucleic acid fragments comprising the regulatory sequences and encoding the tag-sequence, respectively. A 3′-terminal extension was achieved by hybridization with the nucleic acid fragments comprising the regulatory elements. This linear expression construct is further amplified with the help of two terminal primer. These primer comprise the following sequence: 5′-CTTTAAGAAGGAGATATACC+ATG+15-20 bp of the gene-specific sequence (5′-primer, SEQ ID NO: 126) or 5′-ATCGTATGGGTAGCTGGTCCC+TTA+15-20 bp of the gene-specific sequence (3′-primer, SEQ ID NO: 127).

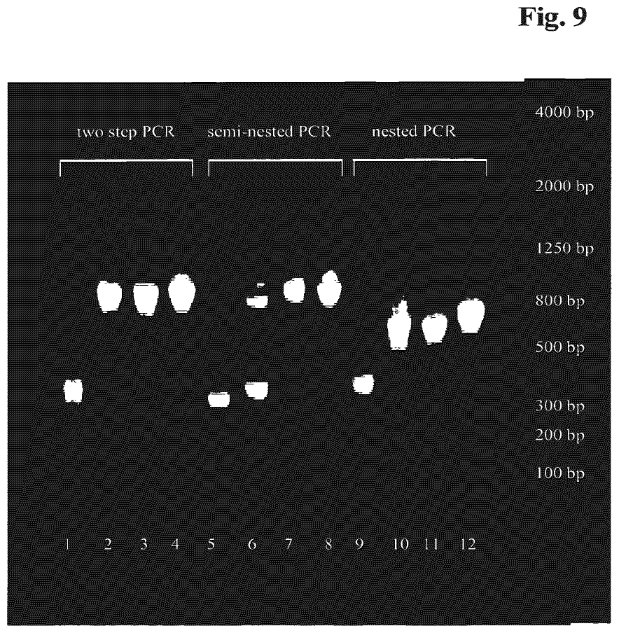

In FIG. 9 lanes 1, 5 and 9 represent the blank water controls. The heavy chain nucleic acid are contained in lanes 4, 8, and 12, and the kappa light chains in lanes 3, 7, and 11. Lanes 2, 6, and 10 show combined samples of both chains. All nucleic acids have the expected size (see Table 38).

TABLE 38
Size of the linear expression constructs.

| immunoglobulin | two fixed primer | one fixed primer | two variable |
|---|---|---|---|
| chain | sets | set | primer sets |
| IgG HC | ~1110 bp | ~1110 bp | ~822 bp |
| IgG LC(κ) | ~1089 bp | ~1089 bp | ~799 bp |

### Example 5

In vitro Translation and huFab Specific ELISA

In vitro translation is carried out as outlined above.

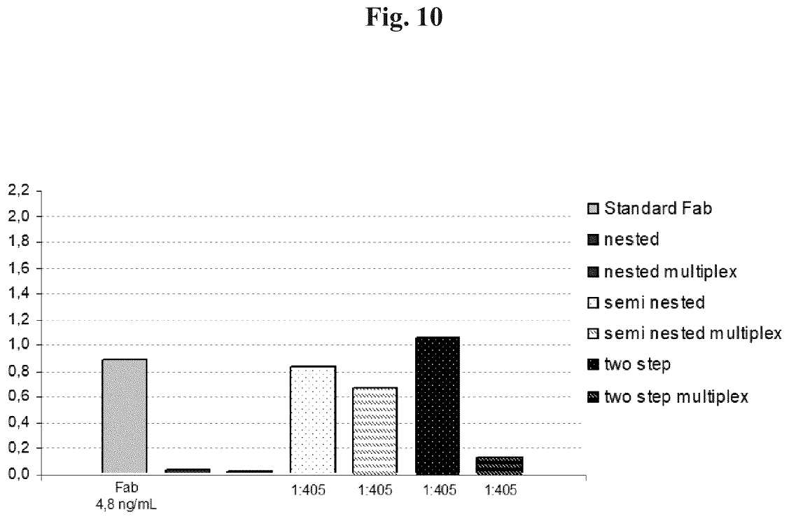

As can be seem from FIG. 10 nucleic acids obtained with a two-step polymerase chain reaction with two variable primer sets does not provide for a linear expression construct which allows the in vitro production of the encoded Fab immunoglobulin fragment. In contrast the two-step polymerase chain reaction with one fixed and one variable set of primer employed in separated successive polymerase chain reactions allows for the subsequent provision of a linear expression construct and the in vitro translation of IgG HC and IgG LC comprising immunoglobulin Fab fragment.

In contrast to this is the two-step polymerase chain reaction comprising one fixed set of primer more efficient in the multiplex format as the polymerase chain reaction employing two fixed sets of primer. By employing only one fixed set of primer up to 5-times higher optical densities can be achieved.

### Example 6

In vitro Translation and huFab Specific ELISA With a Nucleic Acid Obtained From a From Single Cell

### Two-Step Polymerase Chain Reaction With Identical Primer Sets

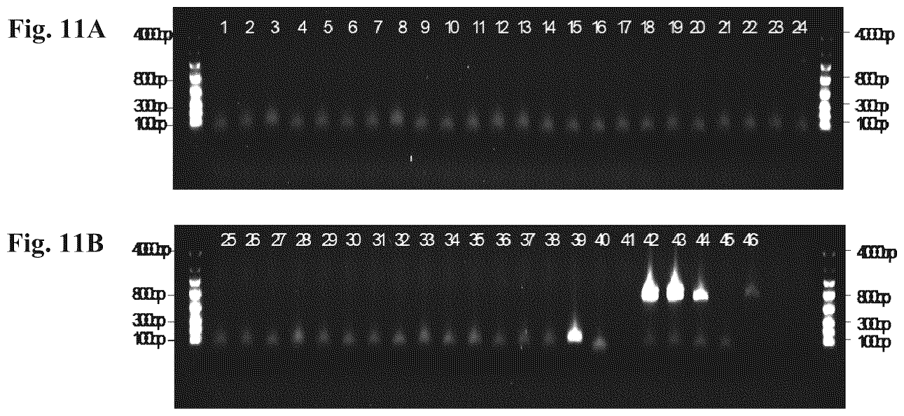

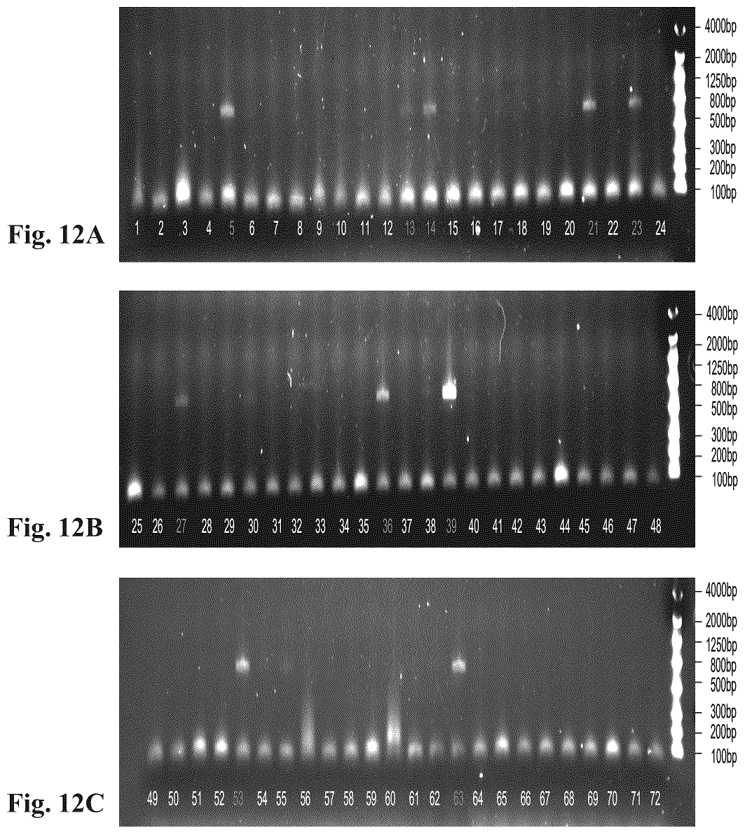

As can be seen from FIGS. 11A-B the control samples yielded no signal in an agarose gel. Single deposited B-cells also showed no signal. IgG HC and IgG LC(κ) could be amplified from control samples.

### Two-Step Polymerase Chain Reaction With One Fixed Set of Primer and One Variable Set of Primer

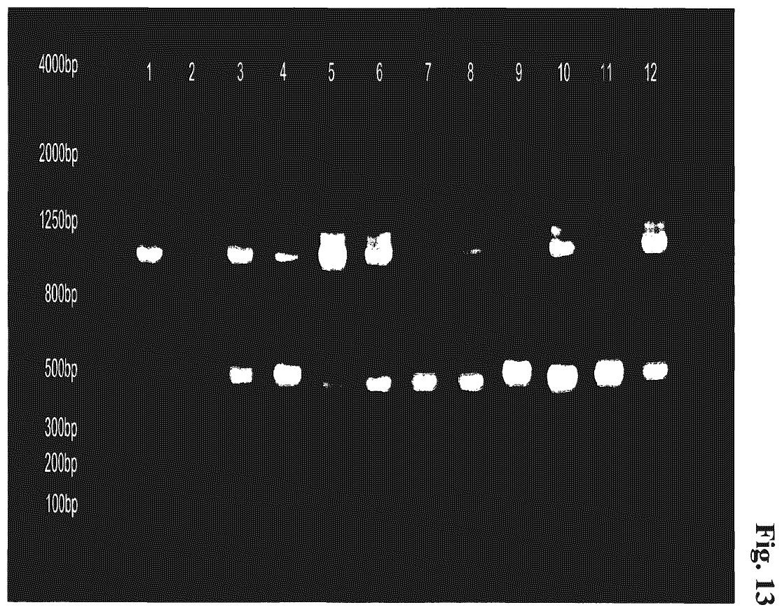

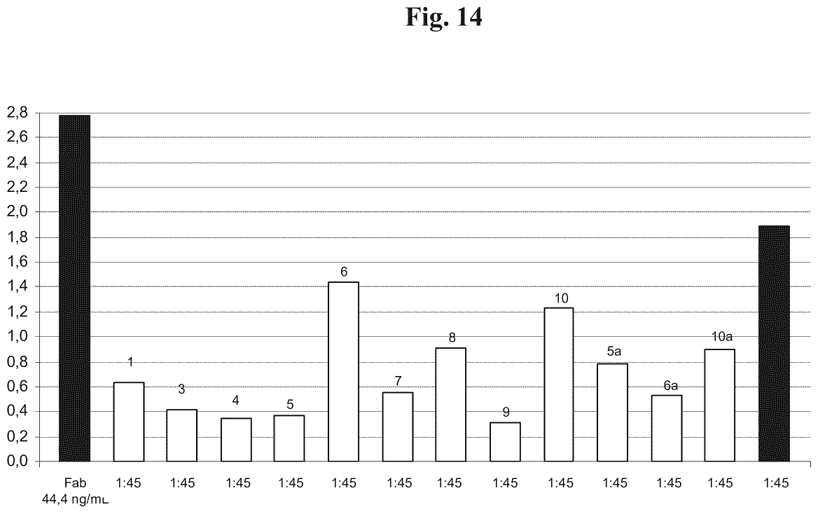

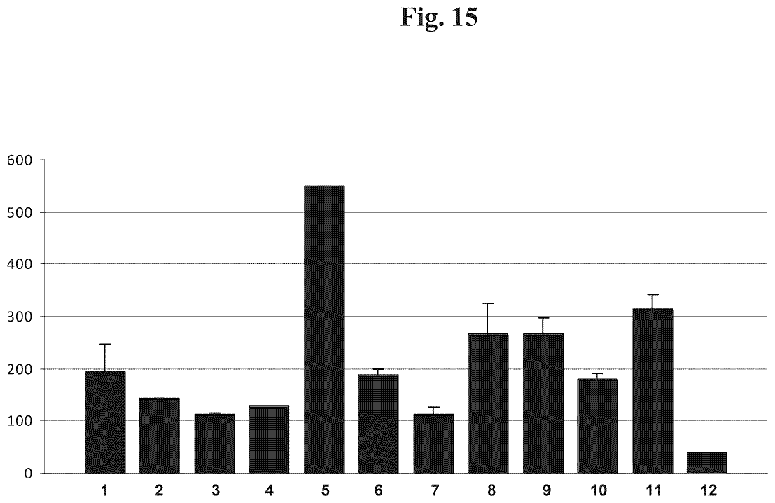

As can be seen from FIG. 13 all nucleic acids, except for sample 2, obtained with a two-step polymerase chain reaction with one fixed set of primer and one variable set of primer allowed for providing a linear expression construct for the production of IgG HC and IgG LC. Thus, these multiplex polymerase chain reaction are well suited.

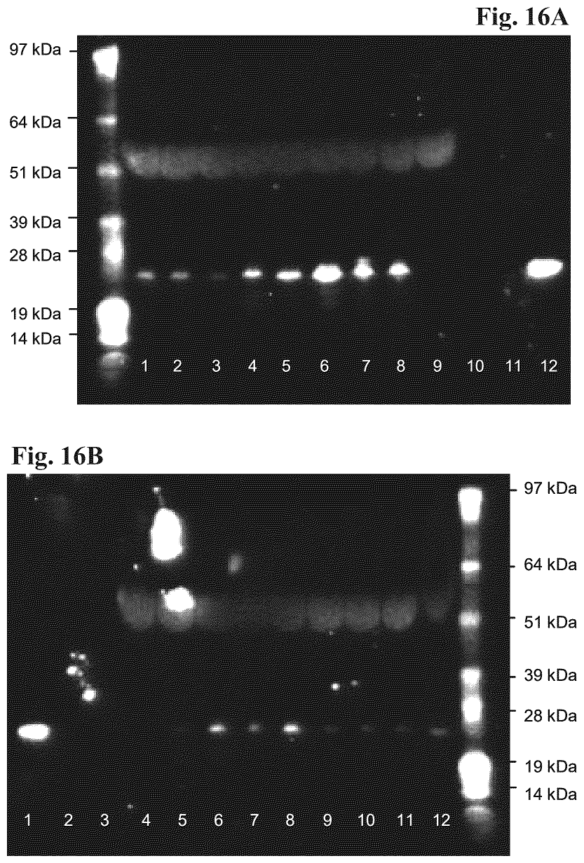

The concentrations of the obtained human kappa Fab immunoglobulin fragments are between 100 and 550 ng/ml. Lanes 9 to 11 of FIGS. 16A-B show IgG HC and IgG LC(κ) obtained from a single cell without the addition of IgG HC positive control. Here the obtained amount of human Fab immunoglobulin fragment was between 180 and 330 ng/ml.

1.-8. (canceled)

9. A method for producing an immunoglobulin comprising the following steps:
(a) providing a single immunoglobulin-producing cell,
(b) obtaining from said cell a nucleic acid encoding an immunoglobulin heavy chain variable domain and
a nucleic acid encoding an immunoglobulin light chain variable domain selected from a kappa light chain variable domain and a lambda light chain variable domain
by a process comprising the steps: (a) performing a first polymerase chain reaction (PCR) to obtain a first PCR product; and (b) performing a second PCR with the first PCR product, whereby the distance of the binding locations of the primers employed in the second PCR is reduced compared to the distance in the first PCR, wherein:
i) for obtaining the nucleic acid encoding the immunoglobulin heavy chain variable domain,
the first PCR is performed
with a set of 5′ primers comprising the nucleic acids of
at least one of SEQ ID NO: 05 and SEQ ID NO: 06,
at least one of SEQ ID NO: 07 and SEQ ID NO: 08,
SEQ ID NO: 09,
at least one of SEQ ID NO: 10 and SEQ ID NO: 11,
SEQ ID NO: 12, and
SEQ ID NO: 13, and
with a 3′ primer or set of primers comprising at least one of the nucleic acids of SEQ ID NO: 104, 105 or 106, and
the second PCR is performed
with a set of 5′ primers comprising the nucleic acids of SEQ ID NOs: 128, 129, 130, 131, 132, 133, 134, 135, 136, 137, 138, 139, 140, and 141 or
with a 5′ primer comprising the nucleic acid of SEQ ID NO: 142; and
with a 3′ primer or set of primers comprising at least one of the nucleic acids of SEQ ID NO: 104, 105, 106 or
with SEQ ID NO: 143,
ii) for obtaining the nucleic acid encoding the immunoglobulin kappa light chain variable domain,
the first PCR is performed
with a set of 5′ primers comprising the nucleic acids of SEQ ID NOs: 16, 17, 18, and 19, and
with a 3′ primer comprising the nucleic acid of SEQ ID NO: 115, and the second PCR is performed
with a set of 5′ primers comprising the nucleic acids of
at least one of SEQ ID NO: 53 or SEQ ID NO: 54,
at least one of SEQ ID NO: 55 or SEQ ID NO: 56,
at least one of SEQ ID NO: 57 or SEQ ID NO: 58,
SEQ ID NO: 59,
SEQ ID NO: 60,
at least one of SEQ ID NO: 61 or SEQ ID NO: 62,
at least one of SEQ ID NO: 63 or SEQ ID NO: 64,
SEQ ID NO: 65,
SEQ ID NO: 66,
SEQ ID NO: 67,
SEQ ID NO: 68,
SEQ ID NO: 69, and
SEQ ID NO: 70, or
SEQ ID NO: 144, and
with a 3′ primer comprising the nucleic acid of SEQ ID NO: 115 or SEQ ID NO: 145, and
iii) for obtaining the nucleic acid encoding the immunoglobulin lambda light chain variable domain,
the first PCR is performed
with a set of 5′ primers comprising the nucleic acids of
SEQ ID NO: 21,
SEQ ID NO: 22, and
at least one of SEQ ID NO: 23, SEQ ID NO: 24, SEQ ID NO: 25 or SEQ ID NO: 26, and
with a 3′ primer or set of primers comprising at least one of the nucleic acids of SEQ ID NO: 120, SEQ ID NO: 121, SEQ ID NO: 122, SEQ ID NO: 123, SEQ ID NO: 124 or SEQ ID NO: 125, and
the second PCR is performed
with a set of 5′ primers comprising the nucleic acids of
SEQ ID NO: 72,
at least one of SEQ ID NO: 73 or SEQ ID NO: 74,
SEQ ID NO: 75,
SEQ ID NO: 76,
at least one of SEQ ID NO: 77 or SEQ ID NO: 78,
SEQ ID NO: 79,
SEQ ID NO: 80,
SEQ ID NO: 81,
at least one of SEQ ID NO: 82 or SEQ ID NO: 83,
at least one of SEQ ID NO: 84 or SEQ ID NO: 85,
SEQ ID NO: 86,
at least one of SEQ ID NO: 87 or SEQ ID NO: 88,
SEQ ID NO: 89,
at least one of SEQ ID NO: 90 or SEQ ID NO: 91, and
SEQ ID NO: 92, and
with a 3′ primer or set of primers comprising at least one of the nucleic acids of SEQ ID NO: 120, SEQ ID NO: 121, SEQ ID NO: 122, SEQ ID NO: 123, SEQ ID NO: 124 or SEQ ID NO: 125;
(c) linking in operable form the nucleic acid encoding the kappa or lambda light chain variable domain with a nucleic acid encoding an immunoglobulin light chain constant domain, and linking in operable form the nucleic acid encoding the heavy chain variable domain with a nucleic acid encoding an immunoglobulin heavy chain constant region into an expressible set of combined nucleic acids,
(d) transfecting a eukaryotic cell with the combined nucleic acids,
(e) cultivating said transfected cell under conditions suitable for the expression of the immunoglobulin, and
(f) recovering the immunoglobulin from the cell or the cultivation medium; thereby producing the immunoglobulin.

10. The method of claim 9, wherein said method comprises a first step of: providing a single immunoglobulin-producing cell and obtaining the mRNA of said cell.

11. The method of claim 10, wherein said method further comprises the following second step of: obtaining cDNA from said mRNA with a reverse transcriptase-PCR.

12. The method of claim 9, wherein the single immunoglobulin-producing cell is a B-cell, a plasmablast, or a plasma cell.

13. The method of claim 9, wherein said immunoglobulin is a human immunoglobulin.

14. The method of claim 9, wherein said immunoglobulin is an immunoglobulin of the class G (IgG).
